SEQUENCE LISTING (3 sequences)

SEQ ID NO: 1 - protein, 1014 aa, Escherichia coli
MGNQWQQKYLLEYNELVSNFPSPERVVSDYIKNCFKTDLPWFSRIDPDNAYFICFSQNRSNSRSYTGWDHLGKYKTEVLTLTQAALINIGYRFDVFDDANSSTGIYKTKSADVFNEENEEKMLPSEYLHFLQKCDFAGVYGKTLSDYWSKYYDKFKLLLKNYYISSALYLYKNGELDEREYNFSMNALNRSDNISLLFFDIYGYYASDIFVAKNNDKVMLFIPGAKKPFLFKKNIADLRLTLKELIKDSDKQQLLSQHFSLYSRQDGVSYAGVNSVLHAIENDGNFNESYFLYSNKTLSNKDVFDAIAISVKKRSFSDGDIVIKSNSEAQRDYALTILQTILSMTPIFDIVVPEVSVPLGLGIITSSMGISFDQLINGDTYEERRSAIPGLATNAVLLGLSFAIPLLISKAGINQEVLSSVINNEGRTLNETNIDIFLKEYGIAEDSISSTNLLDVKLKSSGQHVNIVKLSDEDNQIVAVKGSSLSGIYYEVDIETGYEILSRRIYRTEYNNEILWTRGGGLKGGQPFDFESLNIPVFFKDEPYSAVTGSPLSFINDDSSLLYPDTNPKLPQPTSEMDIVNYVKGSGSFGDRFVTLMRGATEEEAWNIASYHTAGGSTEELHEILLGQGPQSSLGFTEYTSNVNSADAASRRHFLVVIKVHVKYITNNNVSYVNHWAIPDEAPVEVLAVVDRRFNFPEPSTPPDISTIRKLLSLRYFKESIESTSKSNFQKLSRGNIDVLKGRGSISSTRQRAIYPYFEAANADEQQPLFFYIKKDRFDNHGYDQYFYDNTVGLNGIPTLNTYTGEIPSDSSSLGSTYWKKYNLTNETSIIRVSNSARGANGIKIALEEVQEGKPVIITSGNLSGCTTIVARKEGYIYKVHTGTTKSLAGFTSTTGVKKAVEVLELLTKEPIPRVEGIMSNDFLVDYLSENFEDSLITYSSSEKKPDSQITIIRDNVSVFPYFLDNIPEHGFGTSATVLVRVDGNVVVRSLSESYSLNADASEISVLKVFSKKF

SEQ ID NO: 2 - protein, 1014 aa, Escherichia coli
MNVQWQQKYLLEYNELVSNFPSPERVVSDYIRRCFKTDLPWFSQVDPDNTYFIRFSQSRSNSRSYTGWDHLGKYKTGVLTLTQAALINIGYHFDVFDDANASAGIYKTSSADMFNEKNEEKMLPSEYLYFLKGCDFSGIYGRFLSDYWSKYYDKFKLLLKNYYISSALYLYKNGEIDEYEYNFSISALNRRDNISLFFFDIYGYYSSDMFVAKNNERVMLFIPGAKKPFLFEKNIADLRISLKNLIKENDNKQLLSQHFSLYSRQDGITYAGVNSVLNAIENDGVFNESYFLYSNKRINNKDVFDAVAFSVKKRSFSDGDIVIKSNSEAQRDYALTILQTILSMTPIFDVAIPEVSVTLGLGIIASSMGISFDQLINGDTYEERRSAIPGLATNAALLGLSFAIPFLISKAGTNQKILSRYTKHEIRTLNETNIDMFLEEYGINKNSISETKVLEVELKGSGQHVNIVKLSDEDSKIVAVKGNSLSGIYYEVDIETGYEISSRRIYRTEYNDKIFWTRGGGLKGGQSFDFESLKLPIFFKDEPYSAVPGSSLSFINDDSSLLYPNSTPKLPQPTPEMEIVNYVKRAGDFGERLVTLMRGTTEEEAWNIARYHTAGGSTEELHEILLGQGPQSSLGFTEYTSNINSADAASRRHFLVVIKVQVKYINNNNVSHVNHWAIPDEAPVEVLAVVDRRFNFPEPSTPPNISIIHKLLSLRYFKENIESTSRLNLQKLNRGNIDIFKGRGSISSTRQRAIYPYFESANADEQQPVFFYIKKNRFDDFGYDQYFYNSTVGLNGIPTLNTYTGEILSDASSLGSTYWKKYNLTNETSIIRVSNSARGANGIKIALEEVQEGKPVIITSGNLSGCTTIVARKGGYLYKVHTGTTIPLAGFTSTTGVKKAVEVFELLTNNPMPRVEGVMNNDFLVNYLAESFDESLITYSSSEQKIGSKITISRDNVSTFPYFLDNIPEKGFGTSVTILVRVDGNVIVKSLSESYSLNVENSNISVLHVFSKDF

SEQ ID NO: 3 - protein, 1451 aa, Bordetella
MALVGYDGVVEELLALPSEESGDLAGGRAKREKAEFALFGEAPNGDEPIGQDARTWFYYPKYRPVAVSNLKKMQAAIRARLEPESLILQWLIALDVYLGVLIAALSRTAISDLVFEYVKARYEIYYLLNRVPHPLAAAYLKRRRQRPVDRSGRLGSVFEHPLWFAYDELAGTVDLDADIYEQALAESIERRMDGEPDDGSLDTAGHDVWRLCRDGINRGEQAIFQASGPYGVVADAGYMRTVADLAYADALADCLHAQLRIRAQGSVDSPGDEMPRKLDAWEIAKFHLAATQQARVDLLEAAFALDYAALRDVRVYGDYRNALALRFIKREALRLLGARRGNASTMPAVAAGEYDEIVASGAANDAAYVSMAAALIAGVLCDLESAQRTLPVVLARFRPLGVLARFRRLEQETAGMLLGDQEPEPRGFISFTDFRDSDAFASYAEYAAQFNDYIDQYSILEAQRLARILALGSRMTVDQWCLPLQKVRHYKVLTSQPGLIARGIENHNRGIEYCLGRPPLTDLPGLFTMFQLHDSSWLLVSNINGELWSDVLANAEVMQNPTLAALAEPQGRFRTGRRTGGWFLGGPATEGPSLRDNYLLKLRQSNPGLDVKKCWYFGYRQEYRLPAGALGVPLFAVSVALRHSLDDLAAHAKSALYKPSEWQKFAFWIVPFYREIFFSTQDRSYRVDVGSIVFDSISLLASVFSIGGKLGSFTRTQYGNLRNFVVRQRIAGLSGQRLWRSVLKELPALIGASGLRLSRSLLVDLYEIFEPVPIRRLVAGFVSTTTVGGRNQAFLRQAFSAASSSAGRTGGQLASEWRMAGVDATGLVESTSGGRFEGIYTRGLGPLSERTEYFIVESGNAYRVIWDAYTHGWRVVNGRLPPRLTYTVPVRLNGQGHWETHLDVPGRGGAPEIFGRIRTRNLVALAAEQAAPMRRLLNQARRVALRHIDTCRSRLASPRAESDMDAAIRIFFGEPDAGLRQRIGRRLQEVRAYIGDLSPVNDVLYRAGYDLDDVATLFNAVDRNTSLGRQARMELYLDAIVDLHARLGYENARFVDLMAFHLLSLGHAATASEVVEAVSPRLLGNVFDISNVAQLERGIGNPASTGLFVMLGAYSESSPAIFQSFVNDIFPAWRQASGGGPLVWNFGPAAISPTRLDYANTDIGLLNHGDISPLRARPPLGGRRDIDLPPGLDISFVRYDRPVRMSAPRALDASVFRPVDGPVHGYIQSWTGAEIEYAYGAPAAAREVMLTDNVRIISIENGDEGAIGVRVRLDTVPVATPLILTGGSLSGCTTMVGVKEGYLAFYHTGKSTELGDWATAREGVQALYQAHLAMGYAPISIPAPMRNDDLVSIAATYDRAVIAYLGKDVPGGGSTRITRHDAGAGSVVSFDYNAAVQASAVPRLGQVYVLISNDGQGARAVLLAEDLAWAGSGSALDVLNERLVTLFPAPV

(19) United States
(12) Patent Application Publication
(10) Pub. No.: US 2016/0296595 A1
(43) Pub. Date: Oct. 13, 2016

(54) A RHO GTPASE ACTIVATOR FOR USE AS ANTIMICROBIAL AGENT

(71) Applicants: INSERM (INSTITUT NATIONAL DE LA SANTÉ ET DE LA RECHERCHE MÉDICALE), Paris (FR); UNIVERSITE NICE SOPHIA ANTIPOLIS, Nice (FR); CENTRE HOPITALIER UNIVERSITAIRE DE NICE, Nice (FR)

(72) Inventors: Emmanuel LEMICHEZ, Nice (FR); Laurent BOYER, Nice (FR); Anne DOYE, Nice (FR); Pierre MARTY, Nice (FR); Amel METTOUCHI, Nice (FR); Gregory MICHEL, Nice (FR); Patrick MUNRO, Nice (FR)

(21) Appl. No.: 15/036,424

(22) PCT Filed: Nov. 14, 2014
(86) PCT No.: PCT/EP2014/074634

§ 371 Date: May 13, 2016

(30) Foreign Application Priority Data
Nov. 15, 2013 (EP) 13306567.2

(51) Int. Cl. A61K 38/16

(52) U.S. Cl. CPC A61K 38/164

(57) ABSTRACT

The invention relates to a Rho GTPase activator, such as namely the cytotoxic necrotizing factor 1 (CNF1), for use in preventing and/or treating infections by a pathogen in a patient in need thereof. The invention also relates to a Rho GTPase activator, such as CNF1, for use in preventing and/or treating pathologies associated with an infection by a pathogen in a patient in need thereof. For instance, the invention relates to a Rho GTPase activator for use in treating infections by bacteria in a patient in need thereof and also relates to a Rho GTPase activator for use in reducing or eliminating bacteremia in a patient in need thereof.

### FIELD OF THE INVENTION

The invention relates to a Rho GTPase activator, such as namely the cytotoxic necrotizing factor 1 (CNF1), for use in preventing and/or treating infections by a pathogen in a patient in need thereof. The invention also relates to a Rho GTPase activator, such as CNF1, for use in preventing and/or treating pathologies associated with an infection by a pathogen in a patient in need thereof. For instance, the invention relates to a Rho GTPase activator for use in treating infections by bacteria in a patient in need thereof and also relates to a Rho GTPase activator for use in reducing or eliminating bacteremia in a patient in need thereof.

### BACKGROUND OF THE INVENTION

The discovery that mycobacterial extracts promote immune responses to antigens has pioneered the development of immunoadjuvants for vaccination [1]. Discovery of the molecular basis of innate immunity has boosted the development of vaccine adjuvants based on their capacity to stimulate innate immune receptors [2]. The family of bacterial effectors catalysing the activation of Rho proteins has attracted growing attention given their property to trigger immuno-modulator expression [3-7]. It is now established in different model systems that robust activation of Rho GTPases is perceived by cells, as a signal of danger, which is translated into innate immune responses, a phenomenon referred to as microbial effector triggered immunity (ETI) [6]. However, the question of whether ETI can be useful against an intracellular pathogen has never been disclosed nor suggested.

Rho proteins are guanosine triphosphate (GTP)/GDP-based molecular switches, which control cell signalling circuits critical to the dynamic of actin cytoskeleton, cell growth and differentiation, as well as gene expression of immunomodulators [8]. Activation of Rho proteins by bacterial toxins involves the covalent modification of a conserved glutamine residue of these GTPases that is essential for the hydrolysis of guanosine triphosphate (GTP) and their return to a GDP-bound deactivated state [9,10]. CNF1 catalyses the deamidation of the glutamine 61 of Rac1/Cdc42 (Q63 in RhoA) into a glutamic acid, thereby switching Rho proteins into a permanent activated form [9,11,12]. Permanent activation of these GTPases leads to their sensitization to ubiquitin-mediated proteasomal degradation [13-15]. Consequently, CNF1 can be used to trigger a transient activation of Rho GTPases [16].

The small GTPases of the Rho protein family are both a hot spot of posttranslational modifications catalysed by bacterial toxins, and critical sensors of bacterial virulence controlling antimicrobial responses [6,17]. Wide-gene expression analysis of cells treated with CNF1 revealed the expression of a large panel of NF-κB-driven expression of pro-inflammatory cytokines and chemokines [5]. More recent studies have begun to decipher signalling pathways modulated by CNF1 that are involved in innate immune responses [6,7,18]. Modelling E. coli infection in fruit flies has notably revealed that Rac once activated triggers gene expression of antimicrobial peptides via a signalling pathway involving IMD, the Drosophila orthologue of RIP kinase (RIPK) [6,7]. This signalling circuit is sufficient to mount efficient host responses of defence against bacteria that are pathogenic for flies. Consistent with the property of cells to translate CNF1 activity into a protective response against protein antigens are the findings that CNF1 activity stimulates the systemic and mucosal production of IgG and IgA antibodies against ovalbumin and tetanus toxoid [3,5]. Mice immunized against tetanus toxoid together with CNF1, elicit a specific and long-lasting protection against challenge by 10-fold tetanus toxin DL50 [4].

Leishmania infantum/chagasi is the causative agent of visceral leishmaniasis, which is endemic in numerous countries of the south, notably in the Mediterranean basin [19,20]. This disease is fatal if left untreated and represents the second most challenging infectious disease worldwide [20]. Hence, part of the human population is chronically infected with poorly understood consequences on health. A part from the human population, dogs represent the main reservoir and victims. Current treatment is based on chemotherapy with serious limitations such as high cost and toxicity. For these reasons, and on the basis of the robust immunity to reinfection observed in cured patients, several vaccine trials against MVL have been undertaken [21]. Leishmania parasites harness phagocytic cells notably monocytes to survive and replicate. Clinical studies of visceral leishmaniasis implicate the down-modulation of the T-helper Th1 response combined with an increase of Th2 response as the hallmark of the disease [20]. Consistent with this, treatments, which actively increase Th-1 immune responses, promote the clearance of parasites [22].

### SUMMARY OF THE INVENTION

In a first aspect, the invention relates to a Rho GTPase activator for use in preventing and/or treating infections by a pathogen in a patient in need thereof.

In a second aspect, the invention relates to a Rho GTPase activator for use in preventing and/or treating pathologies associated with an infection by a pathogen in a patient in need thereof.

In a third aspect, the invention relates to a pharmaceutical composition comprising a Rho GTPase activator and an antigen derived from a pathogen.

In a fourth aspect, the invention relates to a pharmaceutical composition of the invention for use in preventing and/or treating infections by a pathogen in a patient in need thereof.

In a fifth aspect, the invention relates to a pharmaceutical composition of the invention for use in preventing and/or treating pathologies associated with an infection by a pathogen in a patient in need thereof.

In another aspect, the invention relates to a Rho GTPase activator for use in treating infections by bacteria in a patient in need thereof.

In still another aspect, the invention relates to a Rho GTPase activator for use in reducing bacteremia in a patient in need thereof.

### DETAILED DESCRIPTION OF THE INVENTION

The invention is based on the experimental findings that an activator of Rho GTPases, namely the cytotoxic necrotizing factor 1 (CNF1) can stimulate immune cellular responses against extracellular pathogens (e.g. a bacterium such as Escherichia coli) and intracellular pathogens (e.g. an intracellular protozoan parasite such as Leishmania infantum). The inventors thus demonstrated in a model of intracellular parasite as well as in a model of extracellular bacteria that CNF1 induces the pathogen clearing and reduces therefore the pathogen load (the parasitic load in infected organs and the bacteremia in blood).

Thus, the inventors revealed the capacity of the host to detect Rho activating enzymatic activity of the CNF1 toxin of Escherichia coli during bacteremia in mice. This sensing mechanism was found to potentiate the immune responses triggered by LPS via inflammatory caspases 1/11. The response was protective and increased the host's ability to clear bacteria, thus demonstrating an innate anti-virulence immunity (AVI). They found that AVI triggered by CNF1 works at best with uropathogenic strains of E. coli, which are negative for alpha-hemolysin toxin. Further, they provided evidence that Gr1⁺ cells drove AVI to protect the host during infection. Accordingly, they reported the first example in mice of anti-virulence-triggered immunity induced by a Rho activating factor.

Furthermore, co-administration of the Rho GTPase activating factor CNF1 with an antigen such as for instance Leishmania promastigote antigens at nasal mucosa triggers prophylactic and curative vaccine responses against this parasite. CNF1 activity produced a protection of animals against infection by high inoculum of L. infantum (82% in the spleen and 94.8% in the liver). Moreover, infected animals treated in these conditions showed a marked reduction of parasite burden of 2.3- and 10-fold in the spleen and liver tissues. Analysis of immune parameters by antigen recall established a robust Thelper Th1 polarization of immune memory cells, with a higher production of IL-2 and INF-γ, combined with a decrease of IL-4 production. Thus, CNF1 acts as a potent biological compound eliciting prophylactic and curative vaccinal responses against a model of intracellular parasite.

### Therapeutic Methods and Uses

In a first aspect, the invention relates to a Rho GTPase activator for use in preventing and/or treating infections by a pathogen in a patient in need thereof.

In a second aspect, the invention relates to a Rho GTPase activator for use in preventing and/or treating pathologies associated with an infection by a pathogen in a patient in need thereof.

In one embodiment, the invention relates to a Rho GTPase activator for use in treating infections by bacteria in a patient in need thereof.

In another aspect, the invention relates to a Rho GTPase activator for use in reducing or eliminating bacteremia in a patient in need thereof.

The term “bacteremia” means the presence of bacteria in the blood. Bacteremia can have several consequences. The immune response to the bacteria can cause sepsis and septic shock, which has a relatively high mortality rate. Bacteria can also use the blood to spread to other parts of the body causing metastatic infections away from the original site of infection.

By “Rho GTPase activator”, it is intended herein a compound, which maintains Rho GTPases in a form bound to GTP. By “Rho GTPases”, the one skilled in the art will understand the proteins belonging to the Rho GTPase family, which encompasses RhoA, RhoB, RhoC, Rac1, Rac2 and Cdc42 (Burridge and Wennerberg, 2004 [31]). The level of Rho GTPase bound to GTP can be easily measured by the methods, referred by those skilled in the art as GST-pull down assays and described for RhoA, B and C by Ren et al., 1999 and for Rac1, Rac2 and Cdc42 by Manser et al., 1998 [32].

In one embodiment, said activator is a polypeptide comprising the amino acid sequence starting at the amino acid residue 720 and ending at the amino acid residue 1014 of sequence SEQ ID NO: 1.

In a particular embodiment, said activator is the polypeptide comprising or consisting of the Cytotoxic Necrotizing Factor 1 (CNF1) of sequence SEQ ID NO: 1.

The term “CNF1” has its general meaning in the art and refers to a 114 kDa protein toxin called cytotoxic necrotizing factor 1 (CNF1). The toxin causes alteration of the host cell actin cytoskeleton and promotes bacterial invasion of blood-brain barrier endothelial cells. CNF1 belongs to a unique group of large cytotoxins that cause constitutive activation of Rho guanosine triphosphatases (GTPases), which are key regulators of the actin cytoskeleton. CNF1 consists of an injection domain (amino acid residues 1-719 of SEQ ID NO: 1), allowing the binding and endosomal penetration of the toxin, followed by the intracytoplasmic injection of its catalytic domain (amino acid residues 720-1014 of SEQ ID NO: 1), responsible for Rho GTPases protein family activation. The naturally occurring CNF1 protein has an aminoacid sequence of 1014 amino acids as shown in UniProtKB database under accession number Q47106 and is shown as follows (SEQ ID NO: 1):

MGNQWQQKYLLEYNELVSNFPSPERVVSDYIKNCFKTDLPWFSRIDPDNA
YFICFSQNRSNSRSYTGWDHLGKYKTEVLTLTQAALINIGYRFDVFDDAN
SSTGIYKTKSADVFNEENEEKMLPSEYLHFLQKCDFAGVYGKTLSDYWSK
YYDKFKLLLKNYYISSALYLYKNGELDEREYNFSMNALNRSDNISLLFFD
IYGYYASDIFVAKNNDKVMLFIPGAKKPFLFKKNIADLRLTLKELIKDSD
KQQLLSQHFSLYSRQDGVSYAGVNSVLHAIENDGNFNESYFLYSNKTLSN
KDVFDAIAISVKKRSFSDGDIVIKSNSEAQRDYALTILQTILSMTPIFDI
VVPEVSVPLGLGIITSSMGISFDQLINGDTYEERRSAIPGLATNAVLLGL
SFAIPLLISKAGINQEVLSSVINNEGRTLNETNIDIFLKEYGIAEDSISS
TNLLDVKLKSSGQHVNIVKLSDEDNQIVAVKGSSLSGIYYEVDIETGYEI
LSRRIYRTEYNNEILWTRGGGLKGGQPFDFESLNIPVFFKDEPYSAVTGS
PLSFINDDSSLLYPDTNPKLPQPTSEMDIVNYVKGSGSFGDRFVTLMRGA
TEEEAWNIASYHTAGGSTEELHEILLGQGPQSSLGFTEYTSNVNSADAAS
RRHFLVVIKVHVKYITNNNVSYVNHWAIPDEAPVEVLAVVDRRFNFPEPS
TPPDISTIRKLLSLRYFKESIESTSKSNFQKLSRGNIDVLKGRGSISSTR
QRAIYPYFEAANADEQQPLFFYIKKDRFDNHGYDQYFYDNTVGLNGIPTL
NTYTGEIPSDSSSLGSTYWKKYNLTNETSIIRVSNSARGANGIKIALEEV
QEGKPVIITSGNLSGCTTIVARKEGYIYKVHTGTTKSLAGFTSTTGVKKA
VEVLELLTKEPIPRVEGIMSNDFLVDYLSENFEDSLITYSSSEKKPDSQI
TIIRDNVSVFPYFLDNIPEHGFGTSATVLVRVDGNVVVRSLSESYSLNAD
ASEISVLKVFSKKF

By “injection domain of a Rho GTPase activator” it is intended herein, an amino acid sequence allowing the binding and intracellular penetration of a catalytic domain of a Rho GTPase activator. By “catalytic domain of a Rho GTPase activator” it is intended herein, an amino acid sequence able to activate a Rho GTPase.

The Rho GTPase activator group also includes E. coli cytotoxic necrotizing factor 2 (CNF2, 114 kDa) and dermonecrotic toxins (DNT, 159 kDa) of Bordetella spp. A Rho GTPase activator further encompasses peptides comprising: SOPE SOPE2, IpaC, CagA or the GEF sequence of Dbl as described in the international patent application WO 2005/082408.

In another embodiment, said activator is thus a polypeptide comprising the amino acid sequence starting at the amino acid residue 720 and ending at the amino acid residue 1014 of sequence SEQ ID NO: 2.

In a particular embodiment, said activator is the polypeptide comprising or consisting of the Cytotoxic Necrotizing Factor 2 (CNF2) of sequence SEQ ID NO: 2.

The term “CNF2” has its general meaning in the art and refers to a 114 kDa protein toxin called cytotoxic necrotizing factor 1 (CNF2). As CNF1, CNF2 consists of an injection domain (amino acid residues 1-719 of SEQ ID NO: 2), allowing the binding and endosomal penetration of the toxin, followed by the intracytoplasmic injection of its catalytic domain (amino acid residues 720-1014 of SEQ ID NO: 2), responsible for Rho GTPases protein family activation. The naturally occurring CNF2 protein has an aminoacid sequence of 1014 amino acids as shown in UniProtKB database under accession number C5ZZQ2 and is shown as follows (SEQ ID NO: 2):

MNVQWQQKYLLEYNELVSNFPSPERVVSDYIRRCFKTDLPWFSQVDPDNT
YFIRFSQSRSNSRSYTGWDHLGKYKTGVLTLTQAALINIGYHFDVFDDAN
ASAGIYKTSSADMFNEKNEEKMLPSEYLYFLKGCDFSGIYGRFLSDYWSK
YYDKFKLLLKNYYISSALYLYKNGEIDEYEYNFSISALNRRDNISLFFFD
IYGYYSSDMFVAKNNERVMLFIPGAKKPFLFEKNIADLRISLKNLIKEND
NKQLLSQHFSLYSRQDGITYAGVNSVLNAIENDGVFNESYFLYSNKRINN
KDVFDAVAFSVKKRSFSDGDIVIKSNSEAQRDYALTILQTILSMTPIFDV
AIPEVSVTLGLGIIASSMGISFDQLINGDTYEERRSAIPGLATNAALLGL
SFAIPFLISKAGTNQKILSRYTKHEIRTLNETNIDMFLEEYGINKNSISE
TKVLEVELKGSGQHVNIVKLSDEDSKIVAVKGNSLSGIYYEVDIETGYEI
SSRRIYRTEYNDKIFWTRGGGLKGGQSFDFESLKLPIFFKDEPYSAVPGS
SLSFINDDSSLLYPNSTPKLPQPTPEMEIVNYVKRAGDFGERLVTLMRGT
TEEEAWNIARYHTAGGSTEELHEILLGQGPQSSLGFTEYTSNINSADAAS
RRHFLVVIKVQVKYINNNNVSHVNHWAIPDEAPVEVLAVVDRRFNFPEPS
TPPNISIIHKLLSLRYFKENIESTSRLNLQKLNRGNIDIFKGRGSISSTR
QRAIYPYFESANADEQQPVFFYIKKNRFDDFGYDQYFYNSTVGLNGIPTL
NTYTGEILSDASSLGSTYWKKYNLTNETSIIRVSNSARGANGIKIALEEV
QEGKPVIITSGNLSGCTTIVARKGGYLYKVHTGTTIPLAGFTSTTGVKKA
VEVFELLTNNPMPRVEGVMNNDFLVNYLAESFDESLITYSSSEQKIGSKI
TISRDNVSTFPYFLDNIPEKGFGTSVTILVRVDGNVIVKSLSESYSLNVE
NSNISVLHVFSKDF

In still another embodiment, said activator is thus a polypeptide comprising the amino acid sequence starting at the amino acid residue 1146 and ending at the amino acid residue 1451 of sequence SEQ ID NO: 3.

In a particular embodiment, said activator is the polypeptide comprising or consisting of the DermoNecrotic Toxin (DNT) of sequence SEQ ID NO: 3.

Bordetella dermonecrotic toxin (DNT) has its general meaning in the art and refers to a virulence factor produced by bacteria belonging to the genus Bordetella. The toxin possesses novel transglutaminase activity that catalyzes polyamination or deamidation of the small GTPases of the Rho family. The modified GTPases loose their GTP hydrolyzing activity, function as a constitutive active molecule, and continuously transduce signals to downstream effectors, which mediate the consequent phenotypes of cells intoxicated by DNT. DNT comprises a catalytic domain (amino acid residues 1146-1451 of SEQ ID NO: 3), responsible for Rho GTPases protein family activation. The naturally occurring DNT protein has an amino acid sequence of 1451 amino acids as shown in UniProtKB database under accession number Q45044 and is shown as follows (SEQ ID NO: 3):

MALVGYDGVVEELLALPSEESGDLAGGRAKREKAEFALFGEAPNGDEPI
GQDARTWFYYPKYRPVAVSNLKKMQAAIRARLEPESLILQWLIALDVYL
GVLIAALSRTAISDLVFEYVKARYEIYYLLNRVPHPLAAAYLKRRRQRP
VDRSGRLGSVFEHPLWFAYDELAGTVDLDADIYEQALAESIERRMDGEP
DDGSLDTAGHDVWRLCRDGINRGEQAIFQASGPYGVVADAGYMRTVADL
AYADALADCLHAQLRIRAQGSVDSPGDEMPRKLDAWEIAKFHLAATQQA
RVDLLEAAFALDYAALRDVRVYGDYRNALALRFIKREALRLLGARRGNA
STMPAVAAGEYDEIVASGAANDAAYVSMAAALIAGVLCDLESAQRTLPV
VLARFRPLGVLARFRRLEQETAGMLLGDQEPEPRGFISFTDFRDSDAFA
SYAEYAAQFNDYIDQYSILEAQRLARILALGSRMTVDQWCLPLQKVRHY
KVLTSQPGLIARGIENHNRGIEYCLGRPPLTDLPGLFTMFQLHDSSWLL
VSNINGELWSDVLANAEVMQNPTLAALAEPQGRFRTGRRTGGWFLGGPA
TEGPSLRDNYLLKLRQSNPGLDVKKCWYFGYRQEYRLPAGALGVPLFAV
SVALRHSLDDLAAHAKSALYKPSEWQKFAFWIVPFYREIFFSTQDRSYR
VDVGSIVFDSISLLASVFSIGGKLGSFTRTQYGNLRNFVVRQRIAGLSG
QRLWRSVLKELPALIGASGLRLSRSLLVDLYEIFEPVPIRRLVAGFVST
TTVGGRNQAFLRQAFSAASSSAGRTGGQLASEWRMAGVDATGLVESTSG
GRFEGIYTRGLGPLSERTEYFIVESGNAYRVIWDAYTHGWRVVNGRLPP
RLTYTVPVRLNGQGHWETHLDVPGRGGAPEIFGRIRTRNLVALAAEQAA
PMRRLLNQARRVALRHIDTCRSRLASPRAESDMDAAIRIFFGEPDAGLR
QRIGRRLQEVRAYIGDLSPVNDVLYRAGYDLDDVATLFNAVDRNTSLGR
QARMELYLDAIVDLHARLGYENARFVDLMAFHLLSLGHAATASEVVEAV
SPRLLGNVFDISNVAQLERGIGNPASTGLFVMLGAYSESSPAIFQSFVN
DIFPAWRQASGGGPLVWNFGPAAISPTRLDYANTDIGLLNHGDISPLRA
RPPLGGRRDIDLPPGLDISFVRYDRPVRMSAPRALDASVFRPVDGPVHG
YIQSWTGAEIEYAYGAPAAAREVMLTDNVRIISIENGDEGAIGVRVRLD
TVPVATPLILTGGSLSGCTTMVGVKEGYLAFYHTGKSTELGDWATAREG
VQALYQAHLAMGYAPISIPAPMRNDDLVSIAATYDRAVIAYLGKDVPGG
GSTRITRHDAGAGSVVSFDYNAAVQASAVPRLGQVYVLISNDGQGARAV
LLAEDLAWAGSGSALDVLNERLVTLFPAPV

The term “polypeptide” means herein a polymer of amino acids having no specific length. Thus, peptides, oligopeptides and proteins are included in the definition of “polypeptide” and these terms are used interchangeably throughout the specification, as well as in the claims. The term “polypeptide” does not exclude post-translational modifications that include but are not limited to phosphorylation, acetylation, glycosylation and the like.

A “native sequence” polypeptide refers to a polypeptide having the same amino acid sequence as a polypeptide derived from nature. Thus, a native sequence polypeptide can have the amino acid sequence of naturally-occurring polypeptide from a microorganism such as Escherichia coli. Such native sequence polypeptide can be isolated from nature or can be produced by recombinant or synthetic means. The term “native sequence” polypeptide specifically encompasses naturally-occurring allelic variants of the polypeptide.

A polypeptide “variant” refers to a biologically active polypeptide having at least about 80% amino acid sequence identity with the native sequence polypeptide. Such variants include, for instance, polypeptides wherein one or more amino acid residues are added, or deleted, at the N- or C-terminus of the polypeptide. Ordinarily, a variant will have at least about 80% amino acid sequence identity, more preferably at least about 90% amino acid sequence identity, and even more preferably at least about 95% amino acid sequence identity with the native sequence polypeptide.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% (5 of 100) of the amino acid residues in the subject sequence may be inserted, deleted, or substituted with another amino acid.

In the frame of the present application, the percentage of identity is calculated using a global alignment (i.e., the two sequences are compared over their entire length). Methods for comparing the identity and homology of two or more sequences are well known in the art. The “needle” program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the ebi.ac.uk world wide web site. The percentage of identity in accordance with the invention is preferably calculated using the EMBOSS::needle (global) program with a “Gap Open” parameter equal to 10.0, a “Gap Extend” parameter equal to 0.5, and a Blosum62 matrix.

Polypeptides consisting of an amino acid sequence “at least 80%, 85%, 90%, 95%, 96%, 97%, 98% or 99% identical” to a reference sequence may comprise mutations such as deletions, insertions and/or substitutions compared to the reference sequence. The polypeptide consisting of an amino acid sequence at least 80%, 85%, 90%, 95%, 96%, 97%, 98% or 99% identical to a reference sequence may correspond to an allelic variant of the reference sequence. It may for example only comprise substitutions compared to the reference sequence. The substitutions preferably correspond to conservative substitutions as indicated in the table below.

| Conservative substitutions | Type of Amino Acid |
|---|---|
| Ala, Val, Leu, lle, Met, | Amino acids with aliphatic hydrophobic |
| Pro, Phe, Trp | side chains |
| Ser, Tyr, Asn, Gln, Cys | Amino acids with uncharged but polar |
| | side chains |
| Asp, Glu | Amino acids with acidic side chains |
| Lys, Arg, His | Amino acids with basic side chains |
| Gly | Neutral side chain |

In one embodiment, the polypeptides of the invention may comprise chemical modifications improving their stability and/or their biodisponibility. Such chemical modifications aim at obtaining polypeptides with increased protection of the polypeptides against enzymatic degradation in vivo, and/or increased capacity to cross membrane barriers, thus increasing its half-life and maintaining or improving its biological activity. Any chemical modification known in the art can be employed according to the present invention. Such chemical modifications include but are not limited to:
replacement(s) of an amino acid with a modified and/or unusual amino acid, e.g. a replacement of an amino acid with an unusual amino acid like Nle, Nva or Orn; and/or
modifications to the N-terminal and/or C-terminal ends of the peptides such as e.g. N-terminal acylation (preferably acetylation) or desamination, or modification of the C-terminal carboxyl group into an amide or an alcohol group;
modifications at the amide bond between two amino acids: acylation (preferably acetylation) or alkylation (preferably methylation) at the nitrogen atom or the alpha carbon of the amide bond linking two amino acids;
modifications at the alpha carbon of the amide bond linking two amino acids such as e.g.

acylation (preferably acetylation) or alkylation (preferably methylation) at the alpha carbon of the amide bond linking two amino acids.
chirality changes such as e.g. replacement of one or more naturally occurring amino acids (L enantiomer) with the corresponding D-enantiomers;
retro-inversions in which one or more naturally-occurring amino acids (L-enantiomer) are replaced with the corresponding D-enantiomers, together with an inversion of the amino acid chain (from the C-terminal end to the N-terminal end);
azapeptides, in which one or more alpha carbons are replaced with nitrogen atoms; and/or
betapeptides, in which the amino group of one or more amino acid is bonded to the 0 carbon rather than the a carbon.

As used herein, the term “infection by a pathogen” refers to the detrimental colonization of a host organism by a foreign species. In an infection, the infecting organism seeks to utilize the host's resources to multiply, usually at the expense of the host. The infecting organism interferes with the normal functioning of the host and can lead to chronic wounds, gangrene, loss of an infected limb, and even death.

As used herein, the term “pathologies associated with an infection by a pathogen” relates to the disorders, the diseases or the syndromes which are directly or indirectly a consequence of an infection by said pathogen.

In one embodiment, the pathogen is selected from the group consisting of protozoan parasites, viruses, fungi, and bacteria.

In one embodiment, the pathogen is bacterium.

In a particular embodiment, the bacterium is an extracellular bacterium. In particular embodiment, the bacterium is an intracellular bacterium. In a particular embodiment, the bacterium is selected from the group consisting of Bordetella, Brucella, Campylobacter, Chlamydia, Clostridium, Corynebacterium, Enterococcus, Escherichia, Francisella, Haemophilus, Helicobacter, Legionella, Listeria, Mycobacterium, Neisseria, Pseudomonas, Rickettsia, Salmonella, Shigella, Staphylococcus, Streptococcus, Vibrio and Yersinia.

In another particular embodiment, the bacterium is an antibiotic-resistant bacterium. Infections caused by antibiotic-resistant bacteria represent an overwhelming growing problem both in human and veterinary medicine. For instance, the antibiotic-resistant bacteria encompass methicillin-resistant Staphylococcus aureus (MRSA), community acquired MRSA, a vancomycin-intermediate Staphylococcus aureus (VISA), a vancomycin-resistant Staphylococcus aureus (VRSA) and a glycopeptide-resistant Staphylococcus aureus (GISA).

In a preferred embodiment, the extracellular bacterium is Escherichia Coli.

In another embodiment, the pathogen is an intracellular protozoan parasite

In a particular embodiment, the intracellular protozoan parasite is selected from the group consisting of Leishmania, Trypanosoma, Plasmodium, Toxoplasma, Giardia, Trichomonas and Babesia.

In a preferred embodiment, the intracellular protozoan parasite is Leishmania spp.

Accordingly, the intracellular protozoan parasite Leishmania is selected from the group consisting of Leishmania donovani, Leishmania infantum, Leishmania mexicana, Leishmania amazonesis, Leishmania venezuelensis, Leishmania tropica, Leishmania major, and Leishmania aethiopica.

In a particular embodiment, the pathology is Leishmaniasis.

Leishmaniasis comprises a group of parasitic endemic, or even epidemic, infections widespread in the tropical and subtropical regions of the world. The leishmania, flagellate protozoans of the family Trypansomatidae and the genus Leishmania, are the pathogenic agents responsible for the disease. These parasites infect numerous species of mammals, among which humans and dogs comprise the principal reservoirs of the disease. The leishmanias are transmitted to the different hosts during the infecting bite of phlebotomine sandflies. Nineteen species of leishmanias are potentially capable of infecting humans, and depending on the species of leishmanias involved and factors peculiar to the host (genetic, immunological, etc.), they are the source of very diverse clinical manifestations.

Leishmaniasis develops mainly into three distinct clinical forms: cutaneous, mucocutaneous, and visceral depending on whether the parasites affect the mononuclear phagocytic system of the dermis, the mucous membranes, or the internal organs. The cutaneous lesion can remain localized at the point of inoculation of the parasite and correspond to a benign form with spontaneous healing. Besides this form, more serious pathologies exist, caused by disseminated cutaneous leishmaniasis and mucocutaneous leishmaniasis which are very mutilating and disfiguring. Visceral leishmaniasis affects the mononuclear phagocytic system of numerous organs and tissues, notably the liver, the spleen, and the bone marrow and is fatal in the absence of treatment.

As all vector transmitted diseases, leishmaniasis is characterized by a life cycle that is relatively simple since it is divided between two hosts, mammalian and phlebotomic, and consists of two main forms: a flagellate form called a promastigote, present in the digestive tract of the phlebotomic vector, where it multiplies prior to acquiring its form that is infectious for the mammalian host, also called the metacyclic form; and a non-flagellate form called amastigote, present in the mammalian host, such as dogs and humans.

In another particular embodiment, the intracellular protozoan parasite is selected from the group consisting of Trypanosoma spp. and Plasmodium spp.

In a particular embodiment, the pathologies are selected from the group consisting of malaria and African trypanosomiasis (sleeping sickness).

As used herein, the term “a patient in need thereof” refers to a subject that has been diagnosed with an infection by a pathogen, for instance an intracellular protozoan parasite (such as Leishmania) or a pathology associated with an infection by a pathogen, for instance a pathology associated with an infection by an intracellular protozoan parasite (such as leishmaniasis), or one that is at risk of developing any of these pathology. Such patients may be any mammal, e.g., humans, canines, felidae, and equidae.

Any Rho GTPase activator of the invention as above described may be combined with pharmaceutically acceptable excipients, and optionally sustained-release matrices, such as biodegradable polymers, to form therapeutic compositions.

The invention also relates to a pharmaceutical composition comprising a Rho GTPase activator and a pharmaceutically acceptable excipient.

“Pharmaceutically” or “pharmaceutically acceptable” refers to molecular entities and compositions that do not produce an adverse, allergic or other untoward reaction when administered to a mammal, especially a human, as appropriate. A “pharmaceutically acceptable carrier” or excipient refers to a non-toxic solid, semi-solid or liquid filler, diluent, encapsulating material or formulation auxiliary of any type.

As a general rule, the pharmaceutical composition of the invention is conveniently administered orally, parenterally (subcutaneously, intramuscularly, intravenously, intradermally or intraperitoneally), intrabuccally, intranasally, or transdermally.

Preferably, the pharmaceutical composition is administered to mucosal surface. Still preferably, the pharmaceutical composition is administered intranasally.

The doses used for the administration can be adapted as a function of various parameters, and in particular as a function of the mode of administration used, of the relevant pathology, or alternatively of the desired duration of treatment. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

In another aspect of the invention, there is provided a method of preventing or treating an infection by a pathogen in a patient in need thereof comprising administering a therapeutically effective amount of a Rho GTPase activator of the invention to said patient.

As used herein, the term “therapeutically effective amount” is intended for a minimal amount of active agent, which is necessary to impart prophylactic or therapeutic benefit to a patient. For example, a “therapeutically effective amount of the active agent” to a patient is an amount of the active agent that induces, ameliorates or causes an improvement in the pathological symptoms, disease progression, or physical conditions associated with the disease affecting the patient.

In another aspect of the invention, there is provided a method of preventing or treating a pathology associated with an infection by a pathogen in a patient in need thereof, especially Leishmaniasis, comprising administering a therapeutically effective amount of a Rho GTPase activator of the invention to said patient.

In still another aspect of the invention, there is provided a method of inducing Tₕ1 helper polarization of immune memory cells in a patient in need thereof, comprising administering a therapeutically effective amount of a Rho GTPase activator of the invention to said patient.

In still another aspect of the invention, there is provided a method of reducing the pathogen load in a patient in need thereof, comprising administering a therapeutically effective amount of a Rho GTPase activator of the invention to said patient

In one embodiment, the pathogen load is a parasitic load.

In another embodiment, the pathogen load is bacteremia.

In one embodiment, the invention relates to a pharmaceutical composition comprising a Rho GTPase activator for use in treating infections by bacteria in a patient in need thereof. In one embodiment, the invention relates to a pharmaceutical composition comprising a Rho GTPase activator for use in reducing or eliminating bacteremia in a patient in need thereof.

### Pharmaceutical Compositions

In a third aspect, the invention relates to a pharmaceutical composition comprising a Rho GTPase activator and an antigen derived from a pathogen.

In one embodiment, the antigen is derived from a pathogen is selected from the group consisting of protozoan parasites, viruses, fungi, and bacteria.

In one embodiment, the antigen is derived from an intracellular protozoan parasite.

In a particular embodiment, the antigen derived from an intracellular protozoan parasite is an antigen derived from the group consisting of Leishmania, Trypanosoma, Plasmodium, Toxoplasma, Giardia, Trichomonas and Babesia.

In a preferred embodiment, the antigen derived from an intracellular protozoan parasite is a leishmanial antigen. In another embodiment, the antigen derived from an intracellular protozoan parasite is a mixture of leishmanial antigens. In a particular embodiment, the mixture of leishmanial antigens is a Leishmania promastigote lysate (PL). In another particular embodiment, the mixture of leishmanial antigens is a mixture of Leishmania excreted/secreted proteins (ESPs), such as Leishmania infantum ESPs, as described in the international patent application WO2011/138513.

Any Rho GTPase activator of the invention as above described may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

As a general rule, the pharmaceutical composition of the invention is conveniently administered orally, parenterally (subcutaneously, intramuscularly, intravenously, intradermally or intraperitoneally), intrabuccally, intranasally, or transdermally.

The route of administration contemplated by the invention will depend upon the antigenic substance and the co-formulants. For instance, if the pharmaceutical composition contains saponins, while non-toxic orally or intranasally, care must be taken not to inject the sapogenin glycosides into the blood stream as they function as strong hemolytics. Also, many antigens will not be effective if taken orally.

Preferably, the pharmaceutical composition is administered to mucosal surface. The mucosal surface is selected from the group consisting of mucosal surfaces of the nose, lungs, mouth, eye, ear, gastrointestinal tract, genital tract, vagina, rectum, and the skin. This mode of administration presents a great interest. Indeed, the mucosal membranes contain numerous of dendritic cells and Langerhans cells, which are excellent antigen detecting and antigen presenting cells. The mucosal membranes are also connected to lymphoid organs called mucosal associated lymphoid tissue, which are able to forward an immune response to other mucosal areas. An example of such an epithelium is the nasal epithelial membrane, which consists of practically a single layer of epithelial cells (pseudostratified epithelium) and the mucosal membrane in the upper respiratory tract is connected to the two lymphoid tissues, the adenoids and the tonsils. The extensive network of blood capillaries under the nasal mucosal of the high density of B and T cells, are particularly suited to provide a rapid recognition of the antigen and provide a quick immunological response. Still preferably, the pharmaceutical composition is administered intranasally.

The doses used for the administration can be adapted as a function of various parameters, and in particular as a function of the mode of administration used, of the relevant pathology, or alternatively of the desired duration of treatment. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Pharmaceutical compositions of the invention may comprise an additional therapeutic agent.

In another embodiment, said additional therapeutic active agent is compound or having a bactericide activity.

In another embodiment, said additional therapeutic active agent is compound or vaccine having anti-parasitic activity.

As used herein, the term “compound or vaccine is having anti-parasitic activity” refers to any compound, natural or synthetic, which is used in the course of the treatment of infections by an intracellular protozoan parasite or their pathological consequences. The compound or vaccine having anti-parasitic activity preferably relates to a compound used for decreasing the parasite load in an infected organism. Rho GTPase activators of the invention are useful as adjunctive treatment in parasitic diseases. As such, the association of a Rho GTPase activator and of a compound or vaccine having anti-parasitic activity is advantageous in the frame of the invention since it destroys the parasite itself, while preventing and/or treating consequences of parasitic infection.

In a fourth aspect, the invention relates to a pharmaceutical composition of the invention for use in preventing and/or treating infections by a pathogen in a patient in need thereof. In one embodiment, the invention relates to a pharmaceutical composition of the invention for use in preventing and/or treating infections by intracellular protozoan parasite in a patient in need thereof.

In a fifth aspect, the invention relates to a pharmaceutical composition of the invention for use in preventing and/or treating pathologies associated with an infection by a pathogen in a patient in need thereof. In one embodiment, the invention relates to a pharmaceutical composition of the invention for use in preventing and/or treating pathologies associated with an infection by intracellular protozoan parasite in a patient in need thereof.

In still another aspect, the invention relates to a pharmaceutical composition of the invention for use improving the clinical efficacy of a prophylactic or therapeutic compound or vaccine useful against a pathogen. In one embodiment, the invention relates to a pharmaceutical composition of the invention for use improving the clinical efficacy of a prophylactic or therapeutic compound or vaccine useful against an intracellular protozoan parasite.

As used herein, the term “improving the clinical efficacy” refers to an improvement of the prophylactic or therapeutic effect of a compound or a vaccine and/or the increase of the period of efficacy of said compound or vaccine.

In one embodiment, said vaccine is a mixture of Leishmania excreted/secreted proteins (ESPs), such as Leishmania infantum ESPs, as described in the international patent application WO2011/138513.

The invention will be further illustrated by the following figures and examples. However, these examples and figures should not be interpreted in any way as limiting the scope of the present invention.

### FIGURES

FIG. 1: Antibody responses to L. infantum antigens post-vaccination and infection. Anti-PL IgG antibody responses measured by ELISA post-vaccination (A) and post-infection of vaccinated mice. (B) Groups of seven mice were immunized intranasally with either 3×15 μg plus CNF1 wild-type (PL+WT CNF1) or catalytically inactive CNF1 (PL+mCNF1). Serum samples were tested at 1/100 dilution and revealed using HRP-labelled anti mouse IgG. Interquartile ranges as well as 10-90% percentiles are presented for each group. *: p<0.05. The results are representative from 2 independent experiments.

FIG. 2: Protective effect of nasal immunizations against L. infantum infection. Groups of seven BALB/c mice were immunized either with plus CNF1 wild-type (PL+WT CNF1) or catalytically inactive CNF1 (PL+mCNF1). Fourteen days after the last boost, mice were challenged intraperitonally with 10 stationary phase L. infantum metacyclic parasites. Spleen (A) and liver (B) parasite burdens were quantified 1 month later by ELISA. Bars indicate the mean parasite loads ±SEM. *: p<0.05. The results are representative from 2 independent experiments.

FIG. 3: In vitro antigen recall experiments. Spleen homogenates from mice (seven per groups), immunized by nasal route with PL plus CNF1 wild-type (PL+WT CNF1) or catalytically inactive CNF1 (PL+mCNF1) and next infected with 10⁸ stationary phase L. infantum metacyclic parasites, were challenged with PL at 50 μg/ml for 48 hours. Supernatants were collected and assayed for IFN-γ (A), IL-2 (B) and IL-4 (C) cytokine contents by ELISA. Bars represent the mean cytokine production ±SEM. *: p<0.05.

FIG. 4: CNF1 bears curative immunoadjuvant properties. Groups of five BALB/c mice were immunized by nasal route with PL plus CNF1 wild-type (PL+WT CNF1) or catalytically inactive CNF1 (PL+mCNF1) and next IV infected with 3×10⁸ of stationary phase luciferase parasites. Animals were imaged at days 14, 21 and 28 after inoculation. At day 28, mice were sacrificed and parasite numbers were determined by quantitative PCR using mouse liver (A) and spleen DNA extracts (B). Bars represent the mean cytokine production ±SEM. *: p<0.05

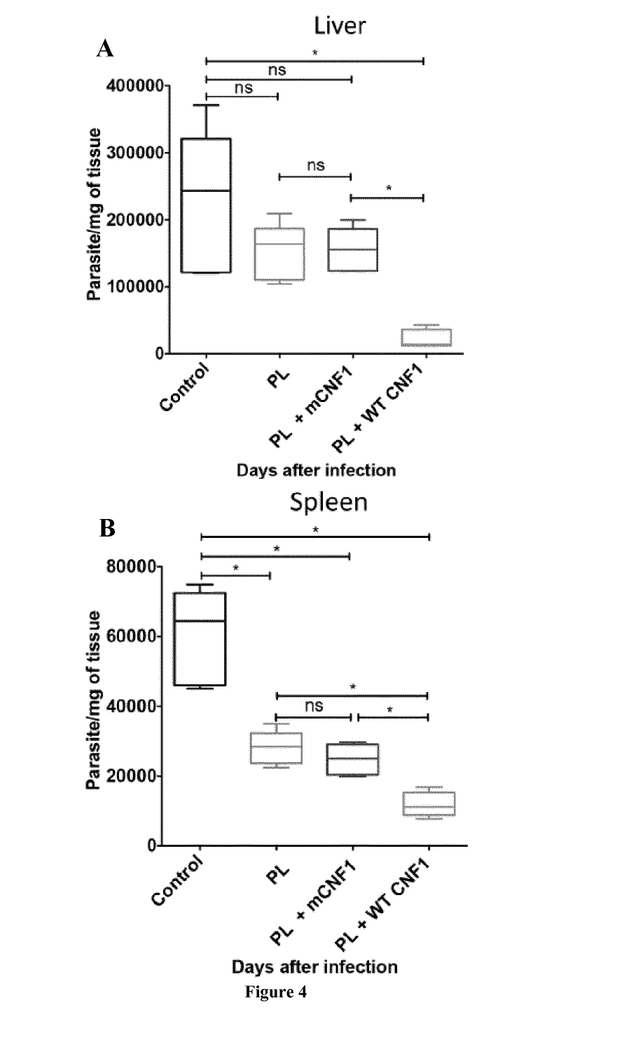

FIG. 5: Escherichia coli-encoded CNF1 toxin triggers bacterial clearing from the blood. Female BALB/c mice were intravenously infected with 10⁷ CFU of Escherichia coli expressing CNF1 or isogenic mutants prior to collection of peripheral blood at 3, 6 or 24 h for bacteremia measurement (n=20-30).

FIG. 6: Infection with E. coli encoding CNF1 triggers clearing of bacteria from the blood and mouse survival. BALB/c mouse survival at 52 h after intravenous injection of 2.10⁸ CFU of E. coli^{CNF+} or the isogenic mutant E. coli^{CNF1-}; n=20. *p<0.05 using a Gehan-Breslow-Wilcoxon chi-squared test.

### EXAMPLE 1

### Mouse Model of Infection by an Intracellular Parasite

Material & Methods

Mice and Ethics Statement:

Six to eight week-old female BALB/c mice were purchased from Charles River (France). Mice were maintained and handled according to the regulations of the European Union, the French Ministry of Agriculture and to FELASA (the Federation of Laboratory Animal Science Associations) recommendations. Experiments were approved by the ethics committee of the Faculté de médecine de Nice, France (Protocol number: 2010-45).

Leishmania infantum Parasites, Antigens and CNF1:

L. infantum MON-1 (MHOM/FR/94/LPN101), was isolated from a patient with mediterranean visceral leishmania contracted in the area of Nice, France. L. infantum promastigotes were routinely grown at 26° C. in Schneider's medium, as previously described [23]. L. infantum clones encoding firefly luciferase were generated as previously described [24].

For promastigote lysate (PL) preparation, stationary phase Leishmania infantum promastigotes were washed and suspended at 10⁹/ml in distilled water [23]. The suspension was submitted to 5 cycles of freeze/thawing to generate a promastigote lysate (PL). Typically 5 mg of Leishmania proteins were obtained from 10⁹ parasites.

Recombinant wild-type cytotoxic necrotizing factor-1 (WT CNF1), as well as its catalytically inactive form (CNF1-C866S; mCNF1) were produced and purified, as previously reported [25]. Both recombinant proteins were passed through a polymixin B column (Affinity pack TM-detoxy gel TM, Pierce) and the lack of endotoxin content was verified using a colorimetric LAL assay (LAL QCL-1000, Cambrex). Each stock of CNF1 preparation (2 mg/ml) was shown to contain less than 0.5 endotoxin units/ml.

Endonasal Immunization and Challenge of BALB/c Mice:

Groups of 7 mice were immunized 3 times at 2-week intervals with 15 μg of promastigote lysate together with 1 μg WT CNF1 or 1 μg catalytically inactive CNF1 (CNF1-C866S: mCNF1). Antigen preparations were delivered at nasal mucosa with a micropipette in 10 μl volumes of PBS (5 μl per nostril). Fourteen days after the last boost, mice were challenged by intraperitoneal route with 10⁸ stationary phase WT or Luciferase L. infantum metacyclic parasites. One month later, mice were sacrificed and aliquots of spleen and liver were collected and analysed for parasite content by ELISA [26].

Analysis of Vaccine-Induced Immune Responses:

To assess total IgG titers, blood samples were recovered from the tail vein after vaccination (one day before infection) and before mice dissection (one month after infection). IgG antibody responses were assessed at 1/100 dilution by ELISA using promastigote lysate (PL)-coated plates, as reported [23].

Vaccine induced cellular immunity was measured post-vaccination using in vitro antigen recall experiments on spleen homogenates as follows: spleen from each individual mouse (5 per group) were homogenized in sterile PBS and erythrocytes were lysed at room temperature using 10 mM NaHCO₃ containing 155 mM NH₄Cl and 0.1 mM EDTA. Spleen cells were then washed twice with PBS, counted and suspended at 5×10⁶ cells/ml in DMEM medium containing 2 mM glutamine, 1 mM sodium pyruvate, 100 U/ml penicillin, 100 μg/ml streptomycin, 50 μM 2-mercaptoethanol and 10% fetal calf serum. Cell suspensions were cultured 48 h in the presence or absence of 50 μg/ml of PL. Supernatants were harvested and assayed for IL-2, IL-4 and IFN-γ content by indirect sandwich ELISA (Pharmingen, Clinisciences, Montrouge, France). The threshold sensitivities of the techniques were in the range of 20-30 pg/ml.

Bioluminescent Analysis of Leishmania infantum Infection:

Each animal (5 mice per group) were infected with 3×10⁸ luciferase parasites. Mice were periodically imaged using the Photon Imager (Biospace Lab, France) system as follows: mice were administered with luciferin (Caliper life science, France) with 300 mg/kg by IP route, and directly, the animals were anesthetized in 5% isoflurane/1 L O₂·min⁻¹ atmosphere. These animals were then placed in the imaging chamber of the Photon Imager. Acquisition of emitted photons, was monitored over a 20 min period

Statistical Analysis:

Non-parametric Mann-Whitney tests were performed using GraphPad Prism version 5.0d for Mac, GraphPad Software, San Diego Calif. USA, www.graphpad.com.

Results

CNF1 Stimulates Humoral Responses Against L. infantum Antigens:

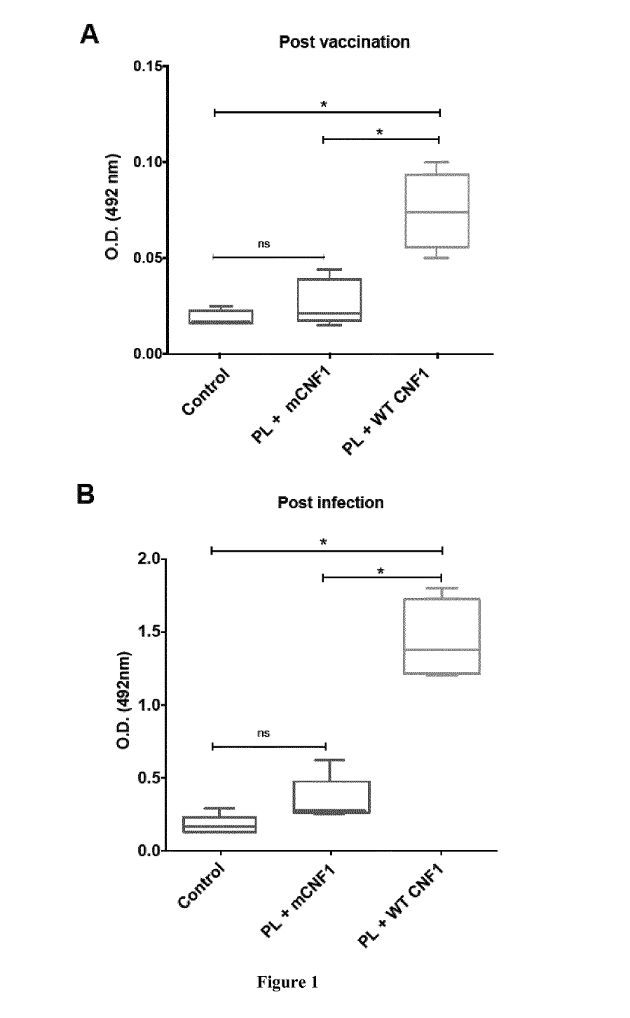

In this study, the inventors were interested to determine the efficacy of CNF1, as immunoadjuvant for induction of protective responses against an intracellular pathogen. In addition, they aimed to evaluate the efficacy of this adjuvant for needle-free vaccination by topic delivery at nasal mucosa. As a model, they choose Leishmania infantum. Groups of 7 mice were immunized 3 times at 2-weeks interval with promastigote lysate (PL), supplemented with either wild-type CNF1 (CNF1, 1 μg: PL+WT CNF1) or the catalytically inactive mutant CNF1-C866S (mCNF1, 1 μg: PL+mCNF1), as a control. A group of naïve mice was also included in order to evaluate the effect of PL+mCNF1. At first, they monitored the adjuvant effect of CNF1 by measure of total seric IgG against PL. In these conditions, they observed a small but reproducible increase of IgG-titers specifically in the serum of mice immunized PL+WT CNF1 (FIG. 1). The inventors concluded that the catalytic active CNF1 specifically primes immune responses against parasite antigens.

CNF1 Confers Protection to Mice Immunized with Leishmania Antigens:

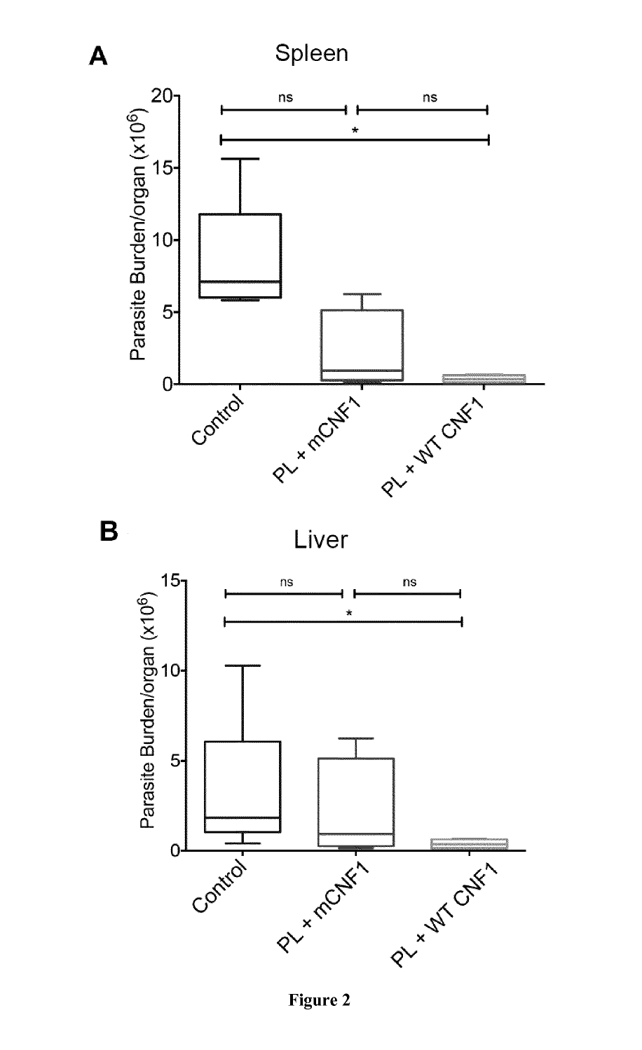

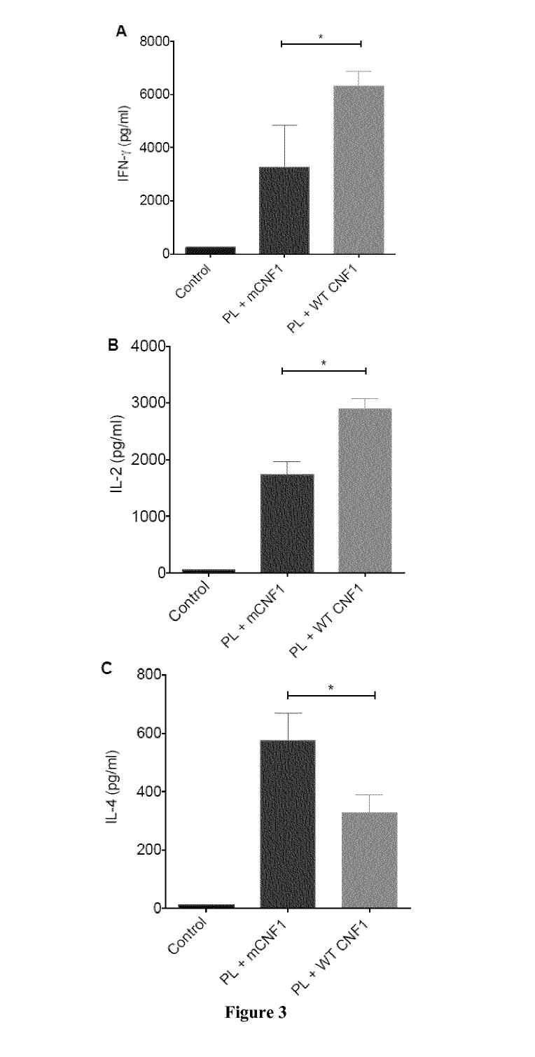

The inventors went on to establish the extent of protection against visceral leishmaniasis in different conditions of immunization. In these experiments, groups of 7 mice were immunized with PL supplemented with either wild-type CNF1 (CNF1, 1 μg: PL+WT CNF1) or the catalytically inactive mutant CNF1-C866S (mCNF1, 1 μg: PL+mCNF1), prior to infection with high doses of infective metacyclic parasites (10⁸ stationary phase). Mice were sacrificed one month later in order to analyse the content of parasites in the spleen and the liver (FIG. 2). In the naïve group, they measured for both types of organs a typical parasite burden ranging from 3 to 8 10⁶ parasites/organ (FIG. 2). The level of parasite alive dramatically decreased in mice that were immunized PL together with wild-type CNF1, as compared to control (23-fold in the spleen and 9-fold in the liver). To evaluate the impact of CNF1 activity, they also quantified levels of parasites in the group of mice immunized PL+mCNF1. Together, our data revealed that the catalytic activity of CNF1 produced a marked increase of 6 fold protection (for spleen and liver), as compared to mCNF1. All these experiments established that mice immunized PL together with active CNF1 have a strong resistance to infection. In parallel, they measured IgG titers against PL in infected mice (FIG. 3). This further revealed a marked increase of 3-fold of the level of total IgG titers in mice immunized PL+WT CNF1, as compared to naïve and PL+mCNF1 conditions (FIGS. 2 and 3). Collectively, these data established that addition of active CNF1 to promastigote lysate confers upon vaccinated mice a resistance to infection by Leishmania infantum.

CNF1 Primes Memory Cell Immunity Against Leishmania infantum:

T cell-mediated (type 1) immune responses confer animals and humans the property to control Leishmania multiplication and dissemination [27]. The protective effect conferred by CNF1 during vaccination against L. infantum was a first indication that this toxin activity might be endowed with T-helper Th1 stimulatory properties. This was assessed on isolated spleen cells by mean of antigen recall. FIG. 3 shows measures of IL-2, INF-γ and IL-4 production recorded after PL-driven antigen recall. No cytokine production was recorded to recall of naïve mice. In contrast, robust cytokine responses were recorded in mice immunized with PL. Interestingly, these responses differ between the different conditions of immunization with catalytically active or inactive CNF1. Optimal IL-2 and IFN-γ memory responses to PL recall were measured for mice immunized PL+WT CNF1, as compared to PL+mCNF1 (1.9 fold increase) (FIG. 3 A-B). In addition, they measured a decrease of IL-4 production of about 2-fold (FIG. 3C). This showed a ratio of IFN-γ/IL-4 of approximately 3.4-fold lower for mice vaccinated with PL+WT CNF1, as compared to mice immunized PL+mCNF1. This profile of immune cell memory responses against PL with an increase of IL-2 and IFN-γ combined with a decrease of IL-4 were indicative that wild-type CNF1 modulates cellular protective responses, thereby conferring an effective protection against Leishmania infection.

CNF1-Primes Direct Cell Immunity Against Leishmania infantum:

Above data unravelled the yet unknown property of CNF1 to stimulate T-helper Th1 immune cell responses. This prompted the inventors to assess whether CNF1 activity might also be endowed with adjuvant curative properties. This was assessed in a model of mice infected with a bioluminescent strain of L. infantum, previously established [24]. Groups of 5 infected mice were immunized at the nasal mucosa with different vaccine compositions. The vaccination was repeated twice at two weeks interval and infection monitored at day 14, 21 and 28 post-infection. Most significantly, this shows that infected mice treated with PL+WT CNF1 dramatically controlled parasite burden, as compared to other vaccine preparation. Indeed, they measured in these conditions a reduction of parasite burden of 82% in the spleen and 94.8% in the liver. They also noticed a modest but reproducible protective effect triggered by Leishmania antigens, as sole vaccine component. Nevertheless, this was largely enhanced by addition of a catalytically active CNF1. In order to get a direct demonstration of the protective effect of PL+WT CNF1, mice were sacrificed at day 28 and the number of parasites directly assessed by qPCR. This revealed the marked curative effect of treatment using a combination of PL together with WT CNF1 with a typical protection of 10-fold measured for the liver and 2.3 fold for the spleen, as compared to pair conditions with catalytically inactive CNF1. Of note, all measurements showed a tight correlation between qPCR and bioluminescence evaluation of parasite burden [24].

Together this series of experiments revealed that the catalytic active form of CNF1 is endowed with adjuvant curative immunoadjuvant properties against visceral Leishmaniasis in a protocol of topic delivery at nasal mucosa.

Discussion:

Previous studies had established that CNF1 activity is a potent adjuvant of humoral responses against protein antigens, thereby conferring long lasting protection against tetanus toxin. The inventors here report the use of CNF1 as immunoadjuvant in the prophylactic and curative vaccination against Leishmania infantum infection. They link this property of CNF1 to its enzymatic activity toward Rho GTPases. They provide evidence by measure of antigen recall that CNF1 activity modulates cellular Th-1 protective responses. This gives molecular insights on how CNF1 treatment confers upon animals a protective effect against infection. This establishes Rho GTPases as targets of great value to stimulate cellular immunity against L. infantum and potentially other intracellular pathogens.

A limited number of vaccine trials against the visceral species L. infantum/chagasi have been reported to date [20,21,28]. Second and third generation vaccine candidates are based on the use of various Leishmania antigen preparations combined with different adjuvants [20]. Second and third generation vaccines using purified or recombinant L. infantum subfractions represent a feasible option for mass vaccination campaigns but their efficacy generally requires the co-administration of an adjuvant [20,21]. Several compounds with adjuvant properties including cytokines, monophosphoryl lipid A, Saponins, C. parvum, P. acnes, Complete Freund Adjuvant have been described in vaccination trials against L. infantum [29]. However, the adjuvant effect of CNF1, a Rho GTPase activating protein, during vaccination against Leishmania species has not yet been reported. Moreover, the immunoadjuvant properties of CNF1 on cellular immunity have not yet been appreciated.

The inventors show that catalytic active CNF1 exerts specifically a protective effect against infection by L. infantum, when mice were immunized at nasal mucosa with a promastigote lysate. Particularly, CNF1 was able to tremendously increase animal resistance to Leishmania infection despite the use of high doses of metacyclic parasites.

In addition to previous reports showing that vaccination with Leishmania antigens confer some protection to animals [30], they here establish that protection is dramatically improved upon addition of catalytic active CNF1.

CNF1 promotes protection against L. infantum infection by molecular mechanisms, which remain to be fully elucidated but involves its catalytic activity toward Rho GTPases. They show here that the adjuvant effect conferred by wild-type CNF1 was accompanied by the elicitation of cellular responses defined by increase secretion of INF-γ and IL-2 cytokines combined with a decrease secretion of IL-4. CNF1 had no effect on the levels of IL-10 production following antigen recall. Production of INF-γ has a major role in eliciting anti-parasite macrophage responses, notably the production of H₂O₂ and induction of NO synthase required for intracellular parasite killing. In addition, IL-2 production and lymphoproliferation contribute to confer a cellular immunoprotection. These protective immune responses are balanced by the immunosupressive responses triggered by the parasite. Immunosupressive cytokines, notably IL-4 is largely involved in the exacerbation of infection by mean of promoting Th-2 responses. Although CNF1 has no effect on IL-10 production, we have measured that catalytic active CNF1 is able to down-modulate the production of IL-4. Thus down-modulation of IL-4 production combined with higher production of INF-γ and IL-2, indicates that CNF1 activity polarizes immune T-cell responses toward a Th-1 compartment.

Collectively, the present data provide a first indication pointing for the property of CNF1 activity to modulate effector T cell responses, conferring animal a protection against L. infantum intracellular parasite.

### EXAMPLE 2

### Mouse Model of Infection by an Extracellular Bacterium

Material & Methods

Mouse Model of Infection:

Female BALB/c mice (6-8 weeks old) were purchased from Charles River (L'Arbresle, France). Mice were injected i.v. with 10⁷ CFU of E. coli. For determination of the bacteremia, blood was collected from the tail vein at 3, 6, 24 and 48 h post-infection, serially diluted in sterile PBS and plated on LB plates containing streptomycin (200 μg/ml) or ampicillin (100 μg/ml) for the strains transformed with a pQE30 derived plasmid, which were incubated for 16 h at 37° C.

Results

CNF1 Toxin Induces Bacterial Clearing from the Blood:

The inventors wished to determine the functional contributions CNF1 to bacterial fitness during sepsis and the ensuing animal death. To clearly address the role of CNF1 during bacteremia they decided to investigate the role of CNF1 without the possible interference of HlyA effects. They thus investigated cnf1 function in the background of deletion mutants of hlyA (referred to as CNF1+) and the double deletion mutant for both hlyA and cnf1 genes (referred to as CNF1).

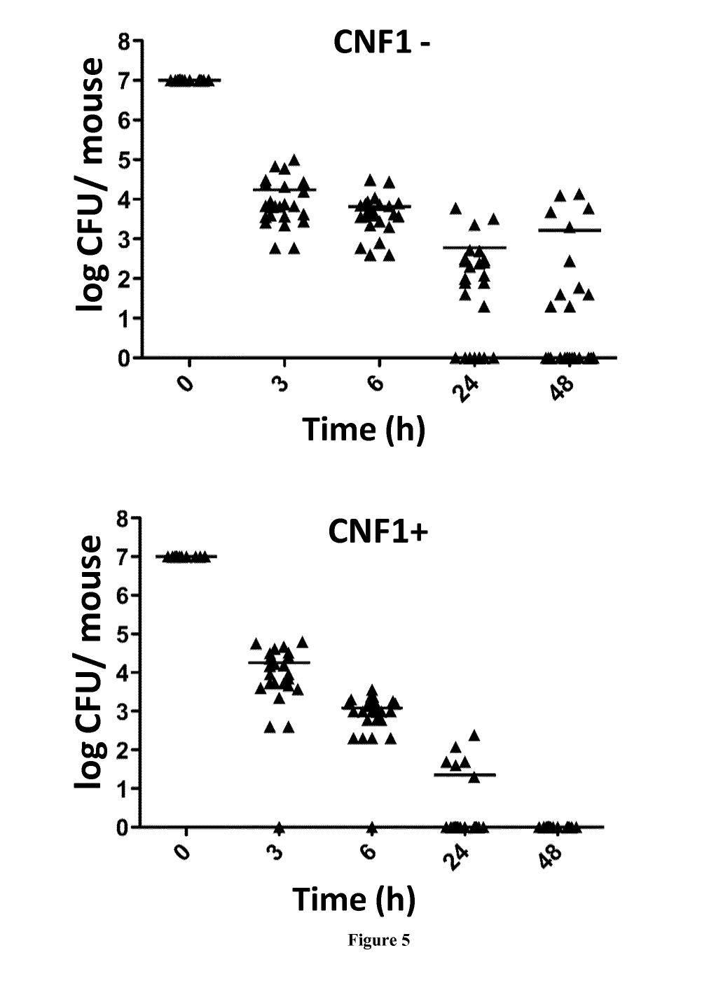

Bacteremia is the most pejorative form of E. coli infection. To analyze the contribution CNF1 during bacteremia mice were infected intravenously with bacteria and kinetics of bacteremia were monitored by serial dilution of blood samples and numeration of CFUs. Notably, the different bacteria were cleared from the blood with very different kinetics. The CNF1+ strain was rapidly cleared, with no bacteria detectable as early as 48 h after infection (FIG. 5) compared to the CNF1− bacteria. Thus, CNF1 appears to be a bacterial factor triggering bacterial clearing from the blood.

This raised the interesting question of whether the rapid clearance of CNF1+ strain was actually due to the enzymatic activity of CNF1. The inventors therefore formally tested this hypothesis by complementing the CNF1− strain with an expression vector of CNF1 (CNF1− pcnf1) or the catalytic inactive mutant CNF1 C866S (CNF1− pcnf1 C866S). Consistent with their hypothesis, CNF1− pcnf1 bacteria were cleared more rapidly from the blood than the CNF1− pcnf1 C866S control strain. Thus, the rapid clearance of bacteria expressing CNF1 relies on the activity of CNF1 and demonstrates that CNF1 increases bacterial eradication from the blood. Further, the present study of the impact of E. coli virulence factors on the bacteria behavior during bacteremia in mice revealed that CNF1 triggers a rapid clearing of E. coli that depend on the CNF1 enzymatic activity.

Material & Methods

Ethics Statement:

This study was carried out in strict accordance with the guidelines of the Council of the European Union (Directive 86/609/EEC) regarding the protection of animals used for experimental and other scientific purposes. The protocol was approved by the Institutional Animal Care and Use Committee on the Ethics of Animal Experiments of Nice, France (reference: NCE/2012-64).

Bacterial Strains and Toxins:

The E. coli UTI89 clinical isolate was originally obtained from a patient with cystitis [33] and was a kind gift from E. Oswald. The UTI89 streptomycin-resistant (SmR) evolved strain (WT) and isogenic mutants were grown in Luria-Bertani (LB) medium supplemented with streptomycin (200 μg/ml). The CNF1 strain was transformed with the pQE30 plasmid (QIAGEN) (E. coli^{CNF1- pempty}), with pQE30-CNF1 (E. coli^{CNF1- pcnf1 WT}) or with pQE30-CNF1 C866S (CNF1− pcnf1 C866S) and grown in LB supplemented with ampicillin (100 μg/ml) plus IPTG (200 μM) for infection experiments. The E. coli^{CNF1+} strain was transformed with pBR322 (E. coli^{CNF1+ pcontrol}) or with pEK50 (plasmid bearing the operon encoding HlyA (hlyCABD) (E. coli^{CNF1+ phlyA}) and grown in LB supplemented with ampicillin (100 μg/ml). The pEK50 plasmid was a kind gift from V. Koronakis. For infections, a 1/50 dilution of an overnight culture of each strain was inoculated and grown to OD600=1.2. Bacteria were either washed in culture medium and diluted to obtain the corresponding MOI for cell culture infection experiments or harvested by centrifugation and washed twice in PBS before dilution in PBS to obtain the desired bacterial concentrations for mouse infection experiments. Recombinant wild-type cytotoxic necrotizing factor-1 (CNF1) and its catalytically inactive form (CNF1-C866S; CNF1 CS) were produced and purified as previously reported [25]. The recombinant proteins were passed through a polymyxin B column (Affinity pack TM-detoxy Gel™, Pierce); the lack of endotoxin content was verified using a colorimetric LAL assay (LAL QCL-1000, Cambrex). Each stock of CNF1 preparation (2 mg/ml) was shown to contain less than 0.5 endotoxin units/ml.

Generation of Isogenic Bacterial Mutant Strains:

The multi-step procedure used to substitute the hlyA and cnf1 genes in the bacterial chromosome was performed as previously described [34]. Briefly, the pMLM135 plasmid (cat, rpsl+) was used to transform the UTI89 streptomycin-resistant (SmR) evolved strain. The integration of pMLM135 into the chromosome was selected by plating cells on chloramphenicol-containing medium at 42° C. Excision of the hlyA or cnf1 gene from the chromosome was selected by plating the cells on medium containing streptomycin (200 μg/ml). The chromosomal deletions were verified by PCR and by monitoring the loss of HlyA and/or CNF1 activity in the deleted strains. We verified that the isogenic mutant strains have growth properties that are identical to those of the UTI89 strain. The sequences of the primers used in this study are available upon request.

Cell Lines and Primary Monocytes:

Murine monocytic cells were obtained from pooled blood from 5-10 mice. Monocytes were isolated using a Ficoll-Paque (GE Healthcare) gradient technique; adherent cells were maintained in M medium (RPMI 1640 medium supplemented with 10% FCS (Lonza), 2 mmol/L L-glutamine, 1 mM pyruvate, 10 mM HEPES, penicillin (100 U/ml), and streptomycin (100 μg/ml). When indicated, GM-CSF was added as previously described [35]. Monocyte isolation was confirmed by flow cytometry analysis using F4/80 and CD11b antibodies (Cedarlane). HEp-2 cells were obtained from ATCC (CCL-23) and maintained according to ATCC instructions.

Mouse Model of Infection:

Female BALB/c and C57BL/6 mice (6-8 weeks old) were purchased from Janvier (Le Genest St Isle, France). Caspase-1/11-impaired (also designated ICE KO) and congenic C57BL/6 mice have been previously described and were kindly provided by R. Flavell [36]. These mice are genetically identical to mice that are now also available from Jackson Laboratories (Stock #016621). Mice were injected i.v. with 10⁷ CFU of E. coli as previously described [37,38]. For determination of bacteremia, blood was collected from the tail vein at indicated times post-infection, serially diluted in sterile PBS and plated on LB plates containing streptomycin (200 μg/ml) or ampicillin (100 μg/ml) for strains transformed with pQE30- or pBR322-derived plasmids, and the plates were incubated for 16 h at 37° C. Injection quality was controlled by plating blood obtained from the mice 5 min after injection. Note that the kinetics for the experiments using the transformed strains were terminated after 24 h because we observed that without selective pressure, the plasmid is stable for up to 24 h. For cytokine analysis, plasma was collected (1200×g, 4° C., 5 min) and stored at −20° C.

In Vivo Gr1⁺ Cell Depletion:

Mice were injected intraperitoneally with a monoclonal anti-Gr1 antibody (RB6-8C5, 100 μg/20 g body weight). After 48 h, the depletion of Gr1⁺ cells was verified in four mice by analyzing F4/80 and/or Gr1-stained white blood cells by flow cytometry. The anti-Gr1-injected mice were then infected with either UTI89 or UTI89 isogenic mutants.

Cytokine Assays:

ELISArrays were performed according to the manufacturer's instructions (QIAGEN, MEM-003A, MEM-004A, MEM-006A, MEM-008A, MEM-009A). Cytokine concentrations were determined by ELISA and by IL-1β maturation visualized by western blotting according to the manufacturer's instructions (KC, TNFαIL-□. R&D Systems, USA; IL-1β, Raybiotech, USA).

Statistical Analyses:

Statistical analysis was performed using Prism V5.0b software (GraphPad, La Jolla, Calif.). Unless stated otherwise, comparisons of two groups were made using the Mann-Whitney nonparametric test and comparisons of three or more groups were made with the Kruskal-Wallis test with Dunn's post-test. P-values<0.05 (*) and P-values<0.01 (**) were considered statistically significant.

Results

CNF1 Activity Decreases Pathogen Load and Favors Host Survival During Bacteremia:

We first assessed the role of CNF1 toxin in determining E. coli burden during the course of bacteremia in the absence of interference from the other toxin, HlyA. For this purpose, we generated both a hlyA deletion mutant (referred to as E. coli^{CNF1+}) and a double hlyA-cnf1− deletion mutant (referred to as E. coli^{CNF1-}). By characterization of the strains at the genetic and functional levels, we determined that the two mutants and the wild-type strain had identical growth properties. BALB/c mice were then infected intravenously with E. coli^{CNF1+} or E. coli^{CNF1-} isogenic strains, and pathogen load was monitored by the serial dilution of blood samples and enumeration of CFUs (FIG. 5). We found that the kinetics of clearance from the bloodstream of these strains were very different. Compared with E. coli^{CNF1-}, which produced 10³ CFU/mouse at 48 h p.i., the E. coli^{CNF1+} strain was rapidly cleared, with no bacteria detectable at 48 h p.i. (FIG. 5). We next assessed whether the rapid clearance of the E. coli^{CNF1+} strain was actually due to the enzymatic activity of CNF1. We tested this hypothesis by complementing the E. coli^{CNF1-} strain with either an expression vector of CNF1 (E. coli^{CNF1- pcnf1}) or an expression vector of the catalytically inactive mutant CNF1 C866S (E. coli^{CNF1- pcnf1 C866S}). E. coli^{CNF1- pcnf1} bacteria were cleared more rapidly from the blood than E. coli^{CNF1- pcnf1 C866S}. Together, these results demonstrate that CNF1 activity promoted the eradication of bacteria from the blood.

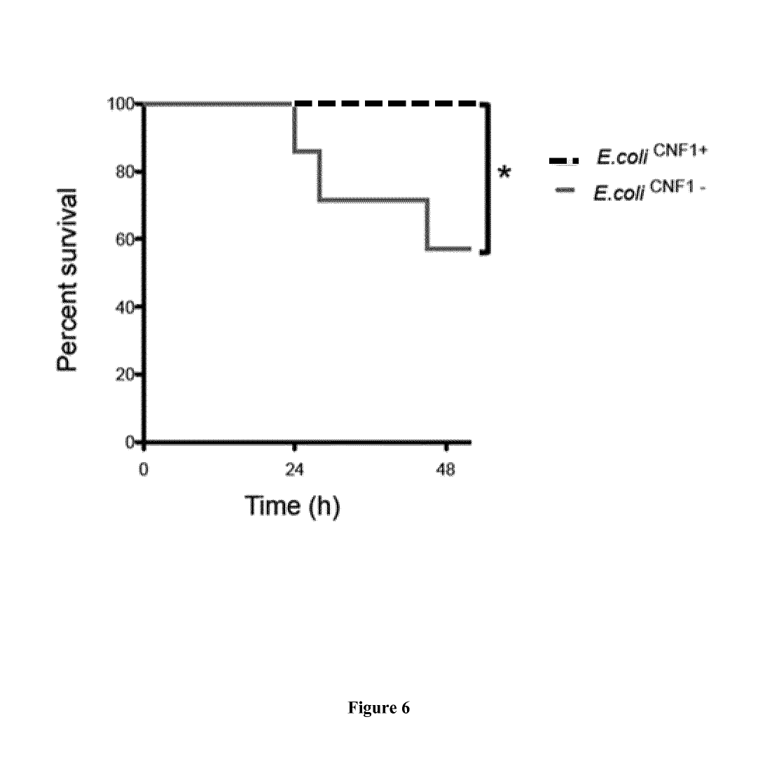

To discern whether there is a link between CNF1 effects on pathogen burden and the virulence of the strains, we monitored the death of animals that had been infected. To this end, E. coli^{CNF1-} bacteria were injected at a dose sufficient to kill half of the mice by 48 h p.i. and compared mouse survival following injection with the different isogenic mutants (FIG. 6). We found that all the mice infected with E. coli^{CNF1+} survived, whereas the group of mice infected with E. coli^{CNF1-} displayed only 57% survival (FIG. 6).

Taken together, our data establish that CNF1 activity has a detrimental effect on bacterial burden in the blood and that it protects against pathogen-induced animal death.

CNF1 Potentiates the LPS-Triggered Secretion of IL-1β in an Inflammatory Caspase-Dependent Manner:

We hypothesized that the CNF1-driven negative impact on bacterial burden involves the modulation of LPS-driven antimicrobial host responses. We assessed this conjecture by profiling the cytokines and chemokines secreted by primary monocytes isolated from the blood of mice after various experimental treatments. The monocytes were challenged with ultrapure LPS, with CNF1 alone, or with a combination of both factors. We used an unbiased approach that utilized an ELISArray semi-quantitative cytokine/chemokine screen that measured the levels of the following factors: IL-1β, TNFα, KC, IL-6, IL-1α, MIP1α, MIP1β, RANTES, MCP1, IL-12, MDC, MIG, IL17, IP10, TARC, EOTAXIN, IL-2, IL-4, IL-5, IL-10, IL-13, IL-23, INFγ, TNFβ1, GM-CSF, and G-CSF. The results show that CNF1 potentiates the LPS-triggered production of the pro-inflammatory cytokines IL-1β, TNFα, and IL-6 primarily, as well as the production of the chemokines MCP1, MIP1α, MIP1β, and KC.

We next performed a quantitative analysis of the impact of CNF1 activity on monocyte responses to LPS. Primary monocytes isolated from the blood of naive mice were treated with endotoxin-free CNF1 or with the catalytically inactive mutant CNF1-C866S. In monocytes intoxicated with recombinant purified CNF1, we recorded a moderate production of KC (75+/−5 pg/ml) that was strictly dependent upon the activity of CNF1. Ultrapure LPS alone or in combination with the catalytically inactive mutant CNF1 C866S triggered a moderate secretion of KC (120+/−10 pg/ml). Strikingly, we observed a 3-fold synergic production of KC (350+/−10 pg/ml) in cells treated with both LPS and CNF1 compared to cells treated with ultrapure LPS alone. The co-stimulation of monocytes with CNF1 and ultrapure LPS resulted in a 12-fold increase in IL-6 secretion, a 2-fold increase in TNFα secretion and a 2-fold increase in IL-1β secretion compared to stimulation with ultrapure LPS alone.

IL-1β is an important mediator of inflammatory responses and is notably important in enabling the host to mount an efficient antibacterial immune response. IL-1β is expressed as a proform that is processed by caspases-1/11 to generate the mature, secreted active form. We further analyzed the effects of the interplay between LPS and CNF1 on IL-1β maturation. This interaction was assessed by immunoblotting to determine the levels of the p17-processed active form of IL-1β that was secreted into the medium upon the co-stimulation of monocytes with LPS+CNF1. Our results confirmed that CNF1 acts at the level of IL-1β maturation/secretion rather than at the level of IL-1β translation. Consistent with the role of CNF1 in promoting caspase-1/11 activity, we observed a complete inhibition of the release of the p17 form of IL-1β in monocytes treated with the pan-caspase inhibitor QVD as well as in monocytes isolated from caspase-1/11 (C1-C11)-impaired mice.

Notably, these results indicate that CNF1 plays a critical role in promoting the caspase-1/11-dependent maturation/secretion of IL-1β by monocytes challenged with LPS.

CNF1 Anti-Virulence Immunity is Mediated by Caspases-1/11:

We assessed the interplay between inflammatory caspases and CNF1 during UPEC-induced bacteremia. To this end, we measured bacterial loads in the blood of C1-C11-impaired mice infected with E. coli^{CNF1+} or E. coli^{CNF1-}. We compared the kinetics of the bacterial burden in these animals to those of their wild-type congenic C57BL/6 littermates. In wild-type animals, we measured a decrease in the bacterial load in animals infected with E. coli^{CNF1+} with no E. coli^{CNF1+} detectable in the blood at 48 h compared to 10⁷ CFU/animal for mice infected with E. coli^{CNF1-}. In contrast to wild-type mice, the E. coli^{CNF1+} burden in C1-C11-impaired mice remained high, with up to 10⁵ CFU/animal at 48 h p.i. These results indicate that inflammatory caspases-1/11 play a major role in the blood clearance of bacteria that is triggered by CNF1 and that these caspases are a major determinant in the control of pathogenic E. coli burden during bacteremia.

In an approach designed to be complementary to our functional approach, we analyzed the role of inflammatory caspases in the initiation of the CNF1-dependent innate immune responses during bacteremia. To this end, we measured the levels of IL-1β and KC cytokines in the sera of C1-C11-impaired mice and their congenic WT littermates at early time points after infection. Wild-type mice infected with E. coli^{CNF1+} displayed higher levels of IL-1β and KC than WT mice infected with E. coli^{CNF1-}. Interestingly, in C1-11-impaired mice infected with E. coli^{CNF1+}, we measured a dramatic decrease in the levels of KC and IL-1β in the sera compared to wild-type mice. This finding is consistent with the fact that inflammatory caspases-1/11 are critical determinants in the CNF1-triggered cytokine response during bacteremia.

Taken together, these results identify caspases-1/11 as a major component of CNF1-induced anti-virulence immunity.

HlyA Counteracts CNF1-Triggered Immunity:

To pinpoint major targets for the development of antimicrobial treatments, we sought to determine how pathogenic bacteria cope with anti-virulence immunity. Although HlyA has been shown to interfere with innate immune responses that occur during urinary tract infections, its role during bacteremia is still unknown. To experimentally address this question, we used the mouse model of bacteremia to analyze the effect of HlyA on the CNF1-triggered protection against bacteremia in mice. We observed that all E. coli strains expressing HlyA displayed higher stability in the blood than other strains, independently of the presence or absence of CNF1. Interestingly, we observed a reduced bacterial burden in the E. coli^{CNF1-} strain compared to the E. coli^{HLY+ CNF1-}, and this reduction in bacterial burden was amplified in the E. coli^{CNF1+} strain. These results suggest that HlyA protects E. coli against the host response, particularly against CNF1-triggered anti-virulence immunity, thereby promoting bacterial stability in the blood. To confirm this possibility, we complemented the E. coli^{CNF1+} strain with a plasmid encoding HlyA (E. coli^{CNF1+ phlyA}). We found that the complementation of E. coli^{CNF1+} with the HlyA expression plasmid stabilized the bacterial load in the blood. We hypothesize that HlyA counteracts the pro-inflammatory effect of CNF1. Consistent with our hypothesis, we measured higher levels of KC in the blood of mice infected with E. coli^{CNF1+} than in the blood of mice infected with E. coli^{CNF1-}. This CNF1-mediated production of KC was abrogated upon infection of the mice with HlyA-expressing strains (E. coli^{HLY+ CNF1+} or E. coli^{HLY+ CNF1-}).

Taken together, our data indicate that HlyA neutralizes CNF1-induced pro-inflammatory cytokine response.

Gr1⁺ Cells are Crucial Effectors of the Anti-E. coli Responses in the Blood.

Next, we aimed to further identify key immune effector cells that control the rapid clearance of E. coli exacerbated by the Rho activating toxin CNF1 during bacteremia. We performed a comparative monitoring of the level of circulating innate immune cells in the blood at early time period of the infection by either E. coli^{CNF1+} strain or the E. coli^{CNF1-} strain. Data are analyzed as percent of CD45 positive cells, a white blood cell marker, to exclude the contamination by red blood cells. We first monitored circulating innate immune cells including monocytes, neutrophils and granulocytes using the CD11b marker. Interestingly, we measured a higher percentage of CD11b⁺/CD45⁺ cells at both 3 h and 6 h p.i. in the blood of mice infected with E. coli CNF1⁺ (3 h: 46% and 6 h: 64%) as compared to the E. coli CNF1⁻ (3 h: 23% and 6 h: 43%). Control mice injected with PBS shows lower level of CD11b⁺/CD45⁺ (3 h: 18% and 6 h: 22%). We found that KC cytokine secretion increased in the sera of mice at 3 h p.i. by CNF1 expressing strains. Because the KC cytokine is involved in chemotaxis as well as in the activation of neutrophils, we hypothesized that the clearance of bacteria is due to cooperation between inflammatory monocytes and neutrophils. To test this hypothesis we monitored in the blood of infected mice the subpopulation of Gr1⁺ cells that includes inflammatory monocytes and neutrophils. Mice infected with E. coli^{CNF1+} shows 37% and 58% of Gr1⁺CD45⁺ cells respectively at 3 h and 6 h as compared only 21% and 40% when mice are infected with E. coli^{CNF1-}, indicating the recruitment of Gr1⁺ cells triggered CNF1. To further demonstrate the key role of Gr1⁺ cells in the clearing of E. coli expressing CNF1 we depleted this subpopulation that includes inflammatory monocytes (Gr1⁺ F4/80⁺) and neutrophils (Gr1⁺ F4/80⁻) prior to infection. We measured an 80% reduction of the Gr1⁺ cell population following injection of anti-Gr1⁺ (Ly-6G) monoclonal antibodies (RB6-8C5). We found that the depletion of Gr1⁺ cells was sufficient to block E. coli clearance during bacteremia and, most importantly, that it prevented the rapid clearance of the E. coli^{CNF1+} strain.

These findings demonstrate the critical role of Gr1⁺ cells in anti-virulence immunity triggered by the Rho GTPase activating toxin CNF1.

Discussion:

The sensing of pathogen-encoded virulence factor activity is emerging as a paradigm of innate immune sensing. However, in vivo proof of the contribution of such sensing to mammalian immunity during infection is still not available. Furthermore, the mechanisms by which pathogenic bacteria cope with the host's capacity to detect their virulence remain to be elucidated. As a major discovery, we demonstrate here the capacity of the host to control bacteremia through the exacerbation of LPS-driven antimicrobial responses upon perception of the CNF1 activity. This host feature relies on inflammatory caspase-1/11 activity and the secretion of pro-inflammatory cytokines, which in turn mobilize Gr1⁺ cells. Importantly, we describe a yet unappreciated role of HlyA in impairing these innate immune responses. Our genetic analysis revealed that by protecting microbes from both CNF1-dependent and CNF1-independent detrimental effects on bacterial burden in the blood, HlyA acts as a major virulence factor during bacteremia. Consistently, pathogen burden and animal death were maximal for HlyA-positive strains.

Inappropriate, excessive or absent innate immune responses have dramatic consequences for human health. Thus, it is critical to decipher how the host determines the pathogenic potential of microbes and responds commensurately. It is currently unclear how AVI systems of detection work together with the recognition of MAMPs such as LPS. Because CNF1 intoxicates cells without additional bacterial factors, it offers a system that can be used to address this critical question. In this work, we report that detection of the CNF1 activity amplifies the cellular LPS response to a large panel of pro-inflammatory cytokines, including IL-1β, by 2- to 12-fold, thereby producing a more potent immune response. The analysis of IL-1β level in the sera of mice infected with E. coli^{CNF1+} indicated that these mice exhibited a 3-fold higher and better resistance to infection than mice infected with isogenic E. coli^{CNF1-}. Our study provides both in vitro and in vivo evidence that AVI works in concert with MAMP-triggered responses to amplify the innate immune response and ultimately improve host viability. We speculate that this cooperation between AVI and MAMP-triggered immunity is a means by which the host gauges the pathogenic potential of microbes and tailors a response commensurate with the estimated threat level.

Sensing of bacterial virulence factor activity has recently emerged as a conserved means of detecting pathogens. Rho GTPases are targeted by various virulence factors encoded by pathogenic bacteria. These virulence factors either post-translationally modify Rho GTPases by deamidation, glucosylation, adenylylation, or ADP-ribosylation or mimic exchange factors or GTPase activating proteins, thus hijacking the GTP/GDP cycle and producing inappropriate activation or inactivation of the critical regulators of these cycles, which are Rho, Rac and Cdc42 GTPases. Interestingly, recent studies indicate that animal hosts have evolved dedicated strategies for detecting the activity of these virulence factors Indeed, based on our work focusing on CNF1 and studies on Salmonella typhimurium SopE/E2 virulence factors, we can speculate that the abnormal activation of Rac/Cdc42 triggers the assembly of an anti-virulence immune complex involving NOD1, RIP kinases, and caspase 1/11-dependent IL-1β maturation during infections. In addition, a recent study implicated the NLR pyrin as a sensor of the inactivation of Rho GTPases by virulence factors via a mechanism that leads to the activation of the pyrin inflammasome and the activation of inflammatory caspase-1. Taken together, these studies indicate that, in parallel to the PRR-detection of MAMPS, the host monitors changes in the GTP/GDP cycle of Rho GTPases rather than monitoring each post-translational modification individually, a process that would require a large repertoire of receptors.

Our observation that CNF1 induces an immune response that is detrimental to bacteria raises the question of why CNF1 has been evolutionarily conserved in the UPEC genome. Several reports have established that the CNF1 toxin can trigger the disruption of epithelial cell junctions, promote cell migration and induce the internalization of bacteria into epithelial cells. One hypothesis is that CNF1 has been evolutionarily conserved as an invasion factor to help bacteria cross epithelia during the early stages of infection. Our results led us to speculate that CNF1 has become genetically associated with HlyA in a manner that protects the bacteria from an otherwise detrimental CNF1-induced innate immune response. Indeed, the CNF1 and HlyA toxins are co-transcribed within a highly conserved PAI, and epidemiological studies have established that CNF1 is always expressed in association with HlyA. Interestingly, the functional relationship between CNF1 and HlyA toxins described here offers a new framework through which to understand the molecular basis of the tight genetic link between these two toxins and that explains why E. coli that express both HlyA and CNF1 are pathogenic to mammals. Consistent with this idea, our work unravels how pathogenic bacteria cope with AVI. We here report a yet unappreciated role of HlyA in the impairment of innate immune responses; in this role, HlyA has major consequences for bacterial burden and for host viability. Our genetic analysis reveals that HlyA protects microbes from both CNF1-dependent and CNF1-independent detrimental effects. Only mice infected with E. coli expressing CNF1 but not HlyA show an increase in KC proinflammatory cytokine level. Given that the level of the bacterial load is minimal under these conditions, this cannot be ascribed to increased LPS exposure. One likely hypothesis is that HlyA targets host immune cells to prevent the production of inflammatory cytokines. Further, we show that bacteria expressing HlyA but not CNF1 show one log unit greater persistence in the blood than bacteria that are deficient in both toxins. Although in our model HlyA acts primarily to counteract the host recognition of CNF1 activity, HlyA most likely has additional effects on other components of the innate immune response to E. coli, including phagocytosis or detection by the immune system of other bacterial components. A common feature of pathogenic bacteria is the production of a wide range of HlyA-like toxins that form pores of various sizes that have specific ionic and molecular selectivities. It will be important to establish which types of pore-forming toxin are able to block innate immunity and to what extent HlyA blocks the recognition of other factors produced by E. coli.

Multicellular organisms have evolved sophisticated defense mechanisms to counter microbial attack. In turn, successful microbial pathogens have evolved strategies to overcome host defenses, leading to the occurrence of diseases or chronic infections. In plants, a similar system of detection of the activity of virulence factors has been termed “effector-triggered immunity” Interestingly, in this model, the pathogen-evolved mechanism counteracting the innate immune defense response has been called a “counter-defense mechanism”. In our model, HlyA counteracts the CNF1-induced host cytokine response. By analogy, if we consider that CNF1 is sensed by the innate immune defense system, HlyA must be considered as a counter-defense effector used by E. coli to counteract the CNF1-induced host response. The data presented in the present work support a model in which HlyA acts as a major virulence factor by protecting microbes from both CNF1-dependent and independent innate immune defenses during bacteremia. Based on this model, RTX toxins might represent a viable drug target for the treatment of UPEC bacteremia.

### REFERENCES

Throughout this application, various references describe the state of the art to which this invention pertains. The disclosures of these references are hereby incorporated by reference into the present disclosure.
1. Freund J, McDermott K (1942) Sensitization to horse serum by means of adjuvants. Proc Soc Exp Biol Med 49: 548-553.
2. Mbow M L, De Gregorio E, Valiante N M, Rappuoli R (2010) New adjuvants for human vaccines. Curr Opin Immunol 22: 411-416.
3. Munro P, Flatau G, Anjuere F, Hofman V, Czerkinsky C et al. (2005) The Rho GTPase activators CNF1 and DNT bacterial toxins have mucosal adjuvant properties. Vaccine 23: 2551-2556.
4. Munro P, Flatau G, Lemichez E (2007) Intranasal immunization with tetanustoxoid and CNF1 as a new mucosal adjuvant protects BALB/c mice against lethal challenge. Vaccine 25: 8702-8706.
5. Munro P, Flatau G, Doye A, Boyer L, Oregioni O et al. (2004) Activation and proteasomal degradation of rho GTPases by cytotoxic necrotizing factor-1 elicit a controlled inflammatory response. J Biol Chem 279: 35849-35857.
6. Stuart L M, Paquette N, Boyer L (2013) Effector-triggered versus pattern triggered immunity: how animals sense pathogens. Nat Rev Immunol 13: 199-206.
7. Boyer L, Magoc L, Dejardin S, Cappillino M, Paquette N et al. (2011) Pathogen-Derived Effectors Trigger Protective Immunity via Activation of the Rac2 Enzyme and the IMD or Rip Kinase Signaling Pathway. Immunity 35: 536-549.
8. Heasman S J, Ridley A J (2008) Mammalian Rho GTPases: new insights into their functions from in vivo studies. Nat Rev Mol Cell Biol 9: 690-701.
9. Lemonnier M, Landraud L, Lemichez E (2007) Rho GTPase-activating bacterial toxins: from bacterial virulence regulation to eukaryotic cell biology. FEMS Microbiol Rev 31: 515-534.
10. Aktories K (2011) Bacterial protein toxins that modify host regulatory GTPases. Nat Rev Microbiol 9: 487-498.
11. Schmidt G, Sehr P, Wilm M, Selzer J, Mann M et al. (1997) Gln 63 of Rho is deamidated by Escherichia coli cytotoxic necrotizing factor-1. Nature 387: 725-729.
12. Flatau G, Lemichez E, Gauthier M, Chardin P, Paris S et al. (1997) Toxin-induced activation of the G protein p21 Rho by deamidation of glutamine. Nature 387: 729-733.
13. Boyer L, Turchi L, Desnues B, Doye A, Ponzio G et al. (2006) CNF1-induced ubiquitylation and proteasome destruction of activated RhoA is impaired in Smurf1−/− cells. Mol Biol Cell 17: 2489-2497.
14. Torrino S, Visvikis O, Doye A, Boyer L, Stefani C et al. (2011) The E3 Ubiquitin-Ligase HACE1 Catalyzes the Ubiquitylation of Active Rac1. Dev Cell 21: 959-965.
15. Doye A, Mettouchi A, Bossis G, Clement R, Buisson-Touati C et al. (2002) CNF1 exploits the ubiquitin-proteasome machinery to restrict Rho GTPase activation for bacterial host cell invasion. Cell 111: 553-564.
16. Visvikis O, Maddugoda M P, Lemichez E (2010) Direct modifications of Rho proteins: deconstructing GTPase regulation. Biol Cell 102: 377-389.
17. Lemichez E, Aktories K (2013) Hijacking of Rho GTPases during bacterial infection. Exp Cell Res
18. Keestra A M, Winter M G, Auburger J J, Frassle S P, Xavier M N et al. (2013) Manipulation of small Rho GTPases is a pathogen-induced process detected by NOD1. Nature 496: 233-237.
19. Marty P, Izri A, Ozon C, Haas P, Rosenthal E et al. (2007) A century of leishmaniasis in Alpes-Maritimes, France. Ann Trop Med Parasitol 101: 563-574.
20. Das A, Ali N (2012) Vaccine Development Against Leishmania donovani. Front Immunol 3: 99.
21. Nagill R, Kaur S (2011) Vaccine candidates for leishmaniasis: a review. Int Immunopharmacol 11: 1464-1488.
22. Fernandes A P, Coelho E A, Machado-Coelho G L, Grimaldi G J, Gazzinelli R T (2012) Making an anti-amastigote vaccine for visceral leishmaniasis: rational, update and perspectives. Curr Opin Microbiol 15: 476-485.
23. Ferrua B, Luci C, Le Fichoux Y, Paul A, Marty P (2006) Imprinting of BALB/c mice with low Leishmania infantum parasite dose markedly protects spleen against high-dose challenge. Vaccine 24: 589-596.
24. Michel G, Ferrua B, Lang T, Maddugoda M P, Munro P et al. (2011) Luciferase-expressing Leishmania infantum allows the monitoring of amastigote population size, in vivo, ex vivo and in vitro. PLoS Negl Trop Dis 5: e1323.
25. Doye A, Boyer L, Mettouchi A, Lemichez E (2006) Ubiquitin-mediated proteasomal degradation of Rho proteins by the CNF1 toxin. Methods Enzymol 406: 447-456.
26. Ferrua B, Le Fichoux Y, Suffia I, Rousseau D, Roptin C et al. (2001) Quantitation of Leishmania infantum in tissues of infected BALB/c mouse by sandwich ELISA. J Immunoassay Immunochem 22: 165-181.
27. Reis A B, Giunchetti R C, Carrillo E, Martins-Filho O A, Moreno J (2010) Immunity to Leishmania and the rational search for vaccines against canine leishmaniasis. Trends Parasitol 26: 341-349.
28. Lemesre J L, Holzmuller P, Cavaleyra M, Goncalves R B, Hottin G et al. (2005) Protection against experimental visceral leishmaniasis infection in dogs immunized with purified excreted secreted antigens of Leishmania infantum promastigotes. Vaccine 23: 2825-2840.
29. Holmgren J, Adamsson J, Anjuere F, Clemens J, Czerkinsky C et al. (2005) Mucosal adjuvants and anti-infection and anti-immunopathology vaccines based on cholera toxin, cholera toxin B subunit and CpG DNA. Immunol Lett 97: 181-188.
30. Roberts M T, Stober C B, McKenzie A N, Blackwell J M (2005) Interleukin-4 (IL-4) and IL-10 collude in vaccine failure for novel exacerbatory antigens in murine Leishmania major infection. Infect Immun 73: 7620-7628.
31. Burridge, K., and Wennerberg, K. (2004). Rho and Rac take center stage. Cell 116, 167-179.
32. Manser, E., Loo, T. H., Koh, C. G., et al., (1998) PAK kinases are directly coupled to the PIX family of nucleotide exchange factors. Mol. Cell vol l pp183-192.
33. Mulvey M A, Schilling J D, Hultgren S J (2001) Establishment of a persistent Escherichia coli reservoir during the acute phase of a bladder infection. Infect Immun 69: 4572-4579.
34. Lemonnier M, Bouet J Y, Libante V, Lane D (2000) Disruption of the F plasmid partition complex in vivo by partition protein SopA. Mol Microbiol 38: 493-505.
35. Jacquel A, Obba S, Boyer L, Dufies M, Robert G et al. (2012) Autophagy is required for CSF-1-induced macrophagic differentiation and acquisition of phagocytic functions. Blood 119: 4527-4531.
36. Kuida K, Lippke J A, Ku G, Harding M W, Livingston D J et al. (1995) Altered cytokine export and apoptosis in mice deficient in interleukin-1 beta converting enzyme. Science 267: 2000-2003.
37. Alteri C J, Mobley H L (2012) Escherichia coli physiology and metabolism dictates adaptation to diverse host microenvironments. Curr Opin Microbiol 15: 3-9.
38. Smith S N, Hagan E C, Lane M C, Mobley H L (2010) Dissemination and systemic colonization of uropathogenic Escherichia coli in a murine model of bacteremia. MBio 1.

1. A method of preventing and/or treating pathogen infections and/or a pathology associated with a pathogen infection in a patient in need thereof, comprising
administering to the patient a therapeutically effective amount of a Rho GTPase activator.

2. (canceled)

3. The method according to claim 1, wherein said Rho GTPase activator is a polypeptide comprising an amino acid sequence starting at amino acid residue 720 and ending at amino acid residue 1014 of sequence SEQ ID NO: 1.

4. The method according to claim 3, wherein said Rho GTPase activator is Cytotoxic Necrotizing Factor 1 (CNF1) of sequence SEQ ID NO: 1.

5. The method of claim 1, wherein the pathogen is selected from the group consisting of protozoan parasites, viruses, fungi, and bacteria.

6. The method according to claim 5, wherein the pathogen is a protozoan parasite.

7. The method according to claim 6, wherein the protozoan parasite is an intracellular protozaon Leishmania parasite.

8. The method according to claim 7, wherein the pathology is Leishmaniasis.

9. A pharmaceutical composition comprising a Rho GTPase activator and an antigen derived from a pathogen.

10. The pharmaceutical composition according to claim 9, wherein said antigen derived from a pathogen is a leishmanial antigen.

11. The pharmaceutical composition according to claim 10, wherein the leishmanial antigen is a Leishmania promastigote lysate.

12. The pharmaceutical composition according to claim 9, wherein said pharmaceutical composition is suitable for administration to a mucosal surface.

13. The pharmaceutical composition according to claim 9, wherein said pharmaceutical composition is suitable for oral administration.

14-15. (canceled)

16. A method of treating bacterial infections and/or for reducing or eliminating bacteremia in a patient in need thereof comprising
administering to the patient a therapeutically effective amount of a Rho GTPase activator.

17. (canceled)

18. The method according to claim 16, wherein said activator is a polypeptide comprising an amino acid sequence starting at amino acid residue 720 and ending at amino acid residue 1014 of sequence SEQ ID NO: 1.

19. The method according to claim 16, wherein said activator is Cytotoxic Necrotizing Factor 1 (CNF1) of sequence SEQ ID NO: 1.

20-21. (canceled)

22. The pharmaceutical composition according to claim 12, wherein said mucosal surface is a nasal surface.

23. The method of claim 1, wherein said Rho GTPase activator is administered as a pharmaceutical composition.

24. The method of claim 16, wherein said Rho GTPase activator is administered as a pharmaceutical composition.
